(19) United States
(12) Patent Application Publication
(10) Pub. No.: US 2022/0175793 A1
(43) Pub. Date: Jun. 9, 2022

(54) THE USE OF AN MGLUR5 ANTAGONIST FOR TREATING OPIOID ANALGESIC TOLERANCE

(71) Applicant: NOVARTIS AG, Basel (CH)

(72) Inventors: Richard Carl Elciario DOLMETSCH, Concord, MA (US); Fabrizio GASPARINI, Basel (CH); Baltazar GOMEZ-MANCILLA, Basel (CH)

(21) Appl. No.: 17/310,287

(22) PCT Filed: Jan. 28, 2020
(86) PCT No.: PCT/IB2020/050637

§ 371 Date: Jul. 27, 2021

Related U.S. Application Data
(60) Provisional application No. 62/798,415, filed on Jan. 29, 2019.

(51) Int. Cl. A61K 31/551; A61K 45/06; A61P 25/04

(52) U.S. Cl. CPC A61K 31/551; A61P 25/04; A61K 45/06

(57) ABSTRACT

The invention relates to the use of a mGluR5 antagonist for the treatment of opioid analgesic tolerance associated with chronic opioid use in chronic pain.

### FIELD OF THE INVENTION

The invention relates to the use of an mGluR5 antagonist for the treatment of opioid analgesic tolerance associated with chronic opioid use in chronic pain.

### BACKGROUND OF THE INVENTION

Treatment of pain is a major healthcare challenge, which requires careful balance between pain control and associated adverse events. In Europe, one-fifth of the population suffers from chronic pain. In addition, diseases associated with pain are rising and furthermore, only one-third to two-thirds of the chronic pain patient population reports >50% pain relief (Trends in Neurosciences, 2014, Vol. 37, No. 3, 146).

Opioids, such as morphine, are potent analgesics used for the treatment of moderate to severe chronic pain of non-malignant or malignant (i.e. cancer) source. However, while medical guidelines recommend the use of opioid therapy for the management of cancer pain, the use thereof for the long-term treatment of non-malignant pain is questionable, in particular due to the many adverse events associated (e.g. nausea, vomiting, pruritus, somnolence, cognitive impairment or dry mouth), the development of analgesic tolerance, and moreover the risks of overdose or opioid use disorder. As stated in the 2016 CDC (Centers for Disease Control and Prevention US Department of Health and Human Services) Guidelines for prescribing opioids for chronic pain, from 1999 to 2014, in the United States, over 165,000 persons died from overdose related to opioid pain medication.

Opioid analgesic tolerance is a well-recognized pharmacological phenomenon associated to opioid therapy, whose underlying biological mechanism is still poorly understood (International Journal of Clinical Pharmacology and Therapeutics, 2004, Vol. 42, No. 4, 191). This phenomenon is characterized by reduced analgesic efficacy, over time, and thus need to increase the opioid dosing in order to maintain analgesic effect. As the dose of an opioid is increased, the potential for side effects is also increased (e.g. respiratory depression, sedation, dizziness, pruritus, nausea, vomiting, constipation, immunologic and hormonal dysfunction), as is the risk for overdose. As reported by the CDC: 1) dosages ≥50 MME/day increase the overdose risk by at least twice the risk at <20 MME/day; 2) in a national sample of Veterans Health Administration patients with chronic pain receiving opioids from 2004-2209, patients who died of overdose were prescribed an average of 98 MME/day. At present, there is no known treatment to reverse opioid analgesic tolerance associated with chronic opioid use in patients with chronic pain. Finding pharmacotherapies for the treatment of opioid analgesic tolerance is thus a high medical need and a major clinical challenge. Morphine tolerance has been treated, for example, with ibudilast, minocycline, fluorocitrate, propentofylline, however, the use of these drugs was associated with significant adverse events. Accordingly, there is a need to identify new therapeutic agents that can be used to treat opioid analgesic tolerance, in particular dugs that can reverse established tolerance, more particularly without increasing adverse effects.

### SUMMARY OF THE INVENTION

The invention relates to the use of an mGluR5 antagonist, for example as defined herein:
in the treatment of opioid analgesic tolerance associated with chronic opioid use in chronic pain;
in the treatment of opioid analgesic tolerance associated with chronic opioid use in chronic pain, the treatment reversing opioid analgesic tolerance;
in a treatment to reverse opioid analgesic tolerance associated with chronic opioid use in chronic pain;
in a treatment to reduce the risk of opioid-related overdose associated with chronic opioid use in chronic pain;
in the treatment of chronic pain in order to reverse opioid analgesic tolerance associated with chronic opioid use;
in the treatment of opioid analgesic tolerance in order to reduce the risk of opioid-related overdose associated with chronic opioid use in chronic pain;
in a treatment to reverse opioid analgesic tolerance associated with chronic opioid use in chronic pain, wherein the opioid dosage [e.g. daily opioid dose; such as >50 mg oral morphine/day or daily equianalgesic dose of another opioid (i.e. morphine milligram equivalent MME/day), such as of from 60 mg oral morphine/day or daily equianalgesic dose of another opioid (e.g. hydrocodine or oxycodone) to 100 mg oral morphine/day or daily equianalgesic dose of another opioid (e.g. hydrocodine or oxycodone)] being administered to the patient is decreased (e.g. by 50% or more, such as by 70% or more, e.g. by 90% or more) and the opioid analgesic effect (i.e. previously achieved by using the higher opioid dosage) is maintained.

### BRIEF DESCRIPTION OF DRAWINGS

FIG. 1: Effects of Compound (III) on foot licking latency

FIG. 2: Effects of Compound (I) on foot licking latency

FIG. 3: Effects of Compound (II) on foot licking latency

### DETAILED DESCRIPTION OF THE INVENTION

It has been found that mavoglurant may be an ideal candidate for treating opioid analgesic tolerance associated with chronic opioid use in chronic pain, having therapeutic advantages, such as one or more of the following:
   i) it reverses opioid analgesic tolerance associated with chronic use of an opioid, for example, compared to placebo: for example, compared to placebo, it reduces the opioid consumption [e.g. a decrease of 50% or more, of the original opioid consumption per day or week, such as a decrease of 70% or more, for example a decrease of 90% or more; for example, as assessed by using biomarkers for metabolites of opioids or as assessed by using patient's self-reported opioid intake (e.g. by using the Timeline Follow Back Opioid Self-reported Diary; e.g. Sobell L C, et al., in The reliability of the timeline followback method applied to drug, cigarette, and cannabis use. Paper presented at the 30th Annual Meeting of the Association for Advancement of Behavior Therapy, New York, N.Y., November 1996) and the analgesic effect is maintained;
   ii) it reduces the opioid consumption and the analgesic effect is maintained, eliminating or reducing (e.g. one or more) withdrawal symptoms associated with opioid use disorder, for example, compared to placebo, for example, as assessed by the Clinical Opiate Withdrawal Scale (COWS; e.g. in Wesson D R, et al., in Journal of Psychoactive Drugs, 2003, Apr-Jun; 35(2): 253-9);
   iii) it reduces the opioid consumption and the analgesic effect is maintained, eliminating or reducing (e.g. one or more) cravings symptoms associated with opioid use, for example, compared to placebo, for example as assessed by the Opioid Craving Scale (OCS; e.g. in McHugh R K et al., Drug and Alcohol Dependency, 2014, Dec. 1; 145: 121-6);
   iv) it reduces the opioid consumption and the analgesic effect is maintained, eliminating or reducing (e.g. one or more) side effects associated with opioid use, for example, compared to placebo, for example as assessed by the Numerical Opioid Side Effect (NOSE; e.g. in Smith H. S., et al., Med Clin North Am., 2007, 91(2):213-228);
   v) it eliminates or reduces (e.g. decrease of 10% or more, such as a decrease of 30% or more) intensity, duration or frequency, of one or more of adverse events associated with chronic use of opioids (e.g. respiratory depression, sedation, dizziness, pruritus, nausea, vomiting, constipation), for example, compared to placebo;
   vi) it reduces the risk of opioid-related overdose associated with chronic opioid use, for example, as assed by the Pasero Opioid-induced Sedation Scale (POSS; e.g. in Pasero C, McCaffery M. Pain Assessment and Pharmacologic Management. St Louis: Mosby/Elsevier; 2011. Section IV, Opioid Analgesics; p.277-622), for example, compared to placebo;
   vii) it improves global functioning, for example as assessed from the Clinical Global Impression Scale-Severity (CGI-S) and Improvement (CGI-I) (Psychiatry, 2007, 4(7): 28-37), for example, compared to placebo;
   viii) it has a favorable therapeutic profile, such as a favorable safety profile or metabolic profile; for example a favorable profile in relation to off-target effects, psychiatric adverse events, toxicity (e.g. genotoxicity) or cardiovascular adverse events (e.g. blood pressure, heart rate, electrocardiography parameters).

Embodiments of the present invention are described herein below:

Embodiments (a):

Embodiment 1a: A mGluR5 antagonist for use in the treatment of opioid analgesic tolerance associated with chronic opioid use in chronic pain.

Embodiment 2a: A mGluR5 antagonist for use in the treatment of opioid analgesic tolerance associated with chronic opioid use in chronic pain, the treatment reversing opioid analgesic tolerance.

Embodiment 3a: A mGluR5 antagonist for use in reversing opioid analgesic tolerance associated with chronic opioid use in chronic pain.

Embodiment 4a: A mGluR5 antagonist for use in a treatment to reduce the risk of opioid-related overdose associated with chronic opioid use in chronic pain.

Embodiment 5a: A mGluR5 antagonist for use in the treatment of chronic pain in order to reverse opioid analgesic tolerance associated with chronic opioid use.

Embodiment 6a: A mGluR5 antagonist for use in the treatment of opioid analgesic tolerance in order to reduce the risk of opioid-related overdose associated with chronic opioid use in chronic pain.

Embodiment 7a: A mGluR5 antagonist for use in reducing opioid consumption in chronic pain.

Embodiment 8a: A mGluR5 antagonist for use in reversing opioid analgesic tolerance associated with chronic opioid use in chronic pain, wherein the opioid dosage [e.g. daily opioid dose] being administered to the patient is decreased (e.g. by 50% or more, such as by 70% or more, e.g. by 90% or more) and the opioid analgesic effect (i.e. previously achieved by using the higher opioid dosage) is maintained.

Embodiment 9a: A mGluR5 antagonist for use according to any one of the preceding embodiments, wherein the mGluR5 antagonist is administered to a patient on an opioid dose of >50 mg oral morphine/day or daily equianalgesic dose of another opioid (i.e. morphine milligram equivalent MME/day), such as a patient on an opioid dose of from 60 to 100 mg oral morphine/day or daily equianalgesic dose of another opioid.

Embodiment 10a: A mGluR5 antagonist for use according to any one of the preceding embodiments, wherein chronic pain is associated to an injury (e.g. wound, burn or fracture) or a disease (e.g. diabetes, multiple sclerosis, arthritis, an autoimmune disease or an infection).

Embodiment 11a: A mGluR5 antagonist for use according to any one of the preceding embodiments, wherein chronic pain is non-malignant chronic pain.

Embodiment 12a: A mGluR5 antagonist for use according to embodiment 11a, wherein non-malignant chronic pain is chronic back pain, such as chronic low back pain, for example with or without previous spine surgery.

Embodiment 13a: A mGluR5 antagonist for use according to any one of the preceding embodiments, wherein the mGluR5 antagonist is administered to an elderly patient.

Embodiment 14a: A mGluR5 antagonist for use according to any one of the preceding embodiments, wherein chronic pain is chronic post-surgical neuropathic pain.

Embodiment 15a: A mGluR5 antagonist for use according to any one of the preceding embodiments, wherein the mGluR5 antagonist is administered in an immediate-release form or a modified-release form; in particular a modified-release form.

Embodiment 16a: A mGluR5 antagonist for use according to any one of the preceding embodiments, wherein the mGluR5 antagonist is administered in the form of a pharmaceutical composition further comprising at least one pharmaceutically acceptable excipient.

Embodiment 17a: A mGluR5 antagonist for use according to any one of the preceding embodiments, wherein the mGluR5 antagonist is administered in combination with one or more further pharmaceutical active ingredient.

Embodiment 18a: A mGluR5 antagonist for use according to any one of the preceding embodiments, wherein the mGluR5 antagonist is selected from the group consisting of Compound (I), Compound (II), and Compound (III); or in each instance a pharmaceutically acceptable salt thereof.

Embodiment 19a: A mGluR5 antagonist for use according to embodiment 18a, wherein the mGluR5 antagonist is Compound (I) or a pharmaceutically acceptable salt thereof.

Embodiment 20a: The mGluR5 antagonist for use according to embodiment 19a, wherein the mGluR5 antagonist is administered in an amount of from 50 mg/b.i.d to 200 mg/b.i.d, in particular 50 mg/b.i.d., 100 mg/b.i.d or 200 mg/b.i.d., such as 200 mg/b.i.d.

Embodiment 21a: A mGluR5 antagonist for use according to embodiment 18a, wherein the mGluR5 antagonist is Compound (II) or a pharmaceutically acceptable salt thereof.

Embodiment 22a: The mGluR5 antagonist for use according to embodiment 21a, wherein the mGluR5 antagonist is administered in an amount of from 1 mg/day to 100 mg/day.

Embodiment 23a: A mGluR5 antagonist for use according to embodiment 18a, wherein the mGluR5 antagonist is Compound (III) or a pharmaceutically acceptable salt thereof.

Embodiment 24a: A combination comprising a mGluR5 antagonist and an opioid.

Embodiment 25a: A combination according to embodiment 24a, wherein the opioid is selected from the group consisting of alphamethylfentanyl, alfentanil, buprenorphine, butorphanol, codeine, diacetylmorphine, dihydrocodeine, dihydroetorphine, dihydromorphine, ethylmorphine, etorphine, fentanyl, hydrocodone, hydromorphone, L-acetylmethadol, levorphanol, methadone, meperidine, morphine, nicomorphine, normethadone, noroxycodone, normorphine, norlevorphanol, oxycodone, oxymorphone, phenazocine, propoxyphene, remifentanil, tramadol, thebaine, tapentadol, levorphanol, sufentanil, pentazocine, carfentanyl, ohmfentanyl, nocaine, ketobemidone, allylprodine, prodine, dextropropoxyphene, dextromoramide, benzitramide, piritramide, dipipanone, loperamide, diphenoxylate, nalbuphine, levomethorpha, dezocine, lefetamine and tildine; in particular the opioid is selected from the group consisting of hydrocodone and oxycodone.

Embodiment 26a: A combination according to embodiments 24a or 25a comprising at least one further active ingredient selected from the group consisting of an antidepressant (e.g. a tricyclic antidepressant, such as amitriptyline, nortriptyline, doxepin, desipramine, imipramine, protriptyline, trimipramine, clomipramine), a serotonin norepinephrine reuptake inhibitor (e.g. duloxetine, venlafaxine, desvenlafaxine, milnacipran, levomilnacipran), a serotonine reuptake inhibitor (e.g. fluoxetine, setraline, paroxetine, fluvoxamine, citalopram, escitalopram, vilazodone, vortioxetine), an anticonvulsant (e.g. gabapentin, pregabalin, carbamazepine, valproic acid, phenytoin, lamotrigine, tiagabine, lacosamide, topiramate, levetiracetam, oxacarbazepine, zonisamide), a non-steroidal anti-inflammatory drug (NSAID; such as naproxen, ibuprofen, meloxicam, diclofenac, fenoprofen, flurbiprofen, diflunisal, etodolac, nabumetone, ketoprofen, piroxicam, sulindac, tolmetin, ketorolac, mefenamic, oxaprozin), a proton pump inhibitor (e.g. omeprazole, pantoprazole, lansoprazole, dexlansoprazole, esomeprazole, rabeprazole), a H2 receptor antagonist (e.g. famotidine, nizatidine, ranitidine, cimetidine), an NMDA inhibitor (e.g. ketamine, amantadine, mematine), NO-NSAID, a COX-2 selective inhibitor, a cannabinoid agonist, a nitric oxide donor, a beta adrenergic agonist, an alpha-2 agonist, a selective prostanoid receptor antagonist, a local anesthetic (e.g. capsaicin, lidocaine), a purinergic P2 receptor antagonist, a neuronal nicotinic receptor agonist, a calcium channel antagonist, a sodium channel blocker (e.g. mexiletine, flecainide), a superoxide dismutase mimetic, a p38 MAP kinase inhibitor, a TRPVI agonist, a glycine receptor antagonist, a corticosteroid, and acetaminophen.

Embodiment 27a: A combination according to any one of the preceding embodiments, wherein the mGluR5 antagonist is selected from the group consisting of Compound (I), Compound (II), and Compound (III); or in each instance a pharmaceutically acceptable salt thereof.

Embodiment 28a: A combination according to embodiment 27a, wherein the mGluR5 antagonist is Compound (I) or a pharmaceutically acceptable salt thereof.

Embodiment 29a: A combination according to embodiment 27a, wherein the mGluR5 antagonist is Compound (II) or a pharmaceutically acceptable salt thereof.

Embodiment 30a: A combination according to embodiment 27a, wherein the mGluR5 antagonist is Compound (III) or a pharmaceutically acceptable salt thereof.

Embodiments (b):

Embodiment 1b: A pharmaceutical composition comprising a mGluR5 antagonist, and at least one pharmaceutically acceptable excipient, for use in the treatment of opioid analgesic tolerance associated with chronic opioid use in chronic pain.

Embodiment 2b: A pharmaceutical composition comprising a mGluR5 antagonist, and at least one pharmaceutically acceptable excipient, for use in the treatment of opioid analgesic tolerance associated with chronic opioid use in chronic pain, the treatment reversing opioid analgesic tolerance.

Embodiment 3b: A pharmaceutical composition comprising a mGluR5 antagonist, and at least one pharmaceutically acceptable excipient, for use in reversing opioid analgesic tolerance associated with chronic opioid use in chronic pain.

Embodiment 4b: A pharmaceutical composition comprising a mGluR5 antagonist, and at least one pharmaceutically acceptable excipient, for use in a treatment to reduce the risk of opioid-related overdose associated with chronic opioid use in chronic pain.

Embodiment 5b: A pharmaceutical composition comprising a mGluR5 antagonist, and at least one pharmaceutically acceptable excipient, for use in the treatment of chronic pain in order to reverse opioid analgesic tolerance associated with chronic opioid use.

Embodiment 6b: A pharmaceutical composition comprising a mGluR5 antagonist, and at least one pharmaceutically acceptable excipient, for use in the treatment of opioid analgesic tolerance in order to reduce the risk of opioid-related overdose associated with chronic opioid use in chronic pain.

Embodiment 7b: A pharmaceutical composition comprising a mGluR5 antagonist, and at least one pharmaceutically acceptable excipient, for use in reducing opioid consumption in chronic pain.

Embodiment 8b: A pharmaceutical composition comprising a mGluR5 antagonist, and at least one pharmaceutically acceptable excipient, for use in reversing opioid analgesic tolerance associated with chronic opioid use in chronic pain, wherein the opioid dosage [e.g. daily opioid dose] being administered to the patient is decreased (e.g. by 50% or more, such as by 70% or more, e.g. by 90% or more) and the opioid analgesic effect (i.e. previously achieved by using the higher opioid dosage) is maintained.

Embodiment 9b: A pharmaceutical composition comprising a mGluR5 antagonist, and at least one pharmaceutically acceptable excipient, for use according to any one of the preceding embodiments, wherein the mGluR5 antagonist is administered to a patient on an opioid dose of >50 mg oral morphine/day or daily equianalgesic dose of another opioid (i.e. morphine milligram equivalent MME/day), such as a patient on an opioid dose of from 60 to 100 mg oral morphine/day or daily equianalgesic dose of another opioid.

Embodiment 10b: A pharmaceutical composition comprising a mGluR5 antagonist, and at least one pharmaceutically acceptable excipient, for use according to any one of the preceding embodiments, wherein chronic pain is associated to an injury (e.g. wound, burn or fracture) or a disease (e.g. diabetes, multiple sclerosis, arthritis, an autoimmune disease or an infection).

Embodiment 11b: A pharmaceutical composition comprising a mGluR5 antagonist, and at least one pharmaceutically acceptable excipient, for use according to any one of the preceding embodiments, wherein chronic pain is non-malignant chronic pain.

Embodiment 12b: A pharmaceutical composition comprising a mGluR5 antagonist, and at least one pharmaceutically acceptable excipient, for use according to embodiment 11b, wherein non-malignant chronic pain is chronic back pain, such as chronic low back pain, for example with or without previous spine surgery.

Embodiment 13b: A pharmaceutical composition comprising a mGluR5 antagonist, and at least one pharmaceutically acceptable excipient, for use according to any one of the preceding embodiments, wherein the mGluR5 antagonist is administered to an elderly patient.

Embodiment 14b: A pharmaceutical composition comprising a mGluR5 antagonist, and at least one pharmaceutically acceptable excipient, for use according to any one of the preceding embodiments, wherein chronic pain is chronic post-surgical neuropathic pain.

Embodiment 15b: A pharmaceutical composition comprising a mGluR5 antagonist, and at least one pharmaceutically acceptable excipient, for use according to any one of the preceding embodiments, wherein the mGluR5 antagonist is administered in an immediate-release form or a modified-release form; in particular a modified-release form.

Embodiment 16b: A pharmaceutical composition comprising a mGluR5 antagonist, and at least one pharmaceutically acceptable excipient, for use according to any one of the preceding embodiments, wherein the mGluR5 antagonist is administered in combination with one or more further pharmaceutical active ingredient.

Embodiment 17b: A pharmaceutical composition comprising a mGluR5 antagonist, and at least one pharmaceutically acceptable excipient, for use according to any one of the preceding embodiments, wherein the mGluR5 antagonist is selected from the group consisting of Compound (I), Compound (II), and Compound (III); or in each instance a pharmaceutically acceptable salt thereof.

Embodiment 18b: A pharmaceutical composition comprising a mGluR5 antagonist, and at least one pharmaceutically acceptable excipient, for use according to embodiment 17b, wherein the mGluR5 antagonist is Compound (I) or a pharmaceutically acceptable salt thereof.

Embodiment 19b: A pharmaceutical composition comprising a mGluR5 antagonist, and at least one pharmaceutically acceptable excipient, for use according to embodiment 18b, wherein the mGluR5 antagonist is administered in an amount of from 50 mg/b.i.d to 200 mg/b.i.d, in particular 50 mg/b.i.d., 100 mg/b.i.d or 200 mg/b.i.d., such as 200 mg/b.i.d.

Embodiment 20b: A pharmaceutical composition comprising a mGluR5 antagonist, and at least one pharmaceutically acceptable excipient, for use according to embodiment 17b, wherein the mGluR5 antagonist is Compound (II) or a pharmaceutically acceptable salt thereof.

Embodiment 21b: A pharmaceutical composition comprising a mGluR5 antagonist, and at least one pharmaceutically acceptable excipient, for use according to embodiment 20b, wherein the mGluR5 antagonist is administered in an amount of from 1 mg/day to 100 mg/day.

Embodiment 22b: A pharmaceutical composition comprising a mGluR5 antagonist, and at least one pharmaceutically acceptable excipient, for use according to embodiment 17b, wherein the mGluR5 antagonist is Compound (III) or a pharmaceutically acceptable salt thereof.

Embodiments (c):

Embodiment 1c: A pharmaceutical combination comprising a mGluR5 antagonist, and at least one further pharmaceutical active ingredient, for use in the treatment of opioid analgesic tolerance associated with chronic opioid use in chronic pain.

Embodiment 2c: A pharmaceutical combination comprising a mGluR5 antagonist, and at least one further pharmaceutical active ingredient, for use in the treatment of opioid analgesic tolerance associated with chronic opioid use in chronic pain, the treatment reversing opioid analgesic tolerance.

Embodiment 3c: A pharmaceutical combination comprising a mGluR5 antagonist, and at least one further pharmaceutical active ingredient, for use in reversing opioid analgesic tolerance associated with chronic opioid use in chronic pain.

Embodiment 4c: A pharmaceutical combination comprising a mGluR5 antagonist, and at least one further pharmaceutical active ingredient, for use in a treatment to reduce the risk of opioid-related overdose associated with chronic opioid use in chronic pain.

Embodiment 5c: A pharmaceutical combination comprising a mGluR5 antagonist, and at least one further pharmaceutical active ingredient, for use in the treatment of chronic pain in order to reverse opioid analgesic tolerance associated with chronic opioid use.

Embodiment 6c: A pharmaceutical combination comprising a mGluR5 antagonist, and at least one further pharmaceutical active ingredient, for use in the treatment of opioid analgesic tolerance in order to reduce the risk of opioid-related overdose associated with chronic opioid use in chronic pain.

Embodiment 7c: A pharmaceutical combination comprising a mGluR5 antagonist, and at least one further pharmaceutical active ingredient, for use in reducing opioid consumption in chronic pain.

Embodiment 8c: A pharmaceutical combination comprising a mGluR5 antagonist, and at least one further pharmaceutical active ingredient, for use in reversing opioid analgesic tolerance associated with chronic opioid use in chronic pain, wherein the opioid dosage [e.g. daily opioid dose] being administered to the patient is decreased (e.g. by 50% or more, such as by 70% or more, e.g. by 90% or more) and the opioid analgesic effect (i.e. previously achieved by using the higher opioid dosage) is maintained.

Embodiment 9c: A pharmaceutical combination comprising a mGluR5 antagonist, and at least one further pharmaceutical active ingredient, for use according to any one of the preceding embodiments, wherein the mGluR5 antagonist is administered to a patient on an opioid dose of >50 mg oral morphine/day or daily equianalgesic dose of another opioid (i.e. morphine milligram equivalent MME/day), such as a patient on an opioid dose of from 60 to 100 mg oral morphine/day or daily equianalgesic dose of another opioid.

Embodiment 10c: A pharmaceutical combination comprising a mGluR5 antagonist, and at least one further pharmaceutical active ingredient, for use according to any one of the preceding embodiments, wherein chronic pain is associated to an injury (e.g. wound, burn or fracture) or a disease (e.g. diabetes, multiple sclerosis, arthritis, an autoimmune disease or an infection).

Embodiment 11c: A pharmaceutical combination comprising a mGluR5 antagonist, and at least one further pharmaceutical active ingredient, for use according to any one of the preceding embodiments, wherein chronic pain is non-malignant chronic pain.

Embodiment 12c: A pharmaceutical combination comprising a mGluR5 antagonist, and at least one further pharmaceutical active ingredient, for use according to embodiment 11b, wherein non-malignant chronic pain is chronic back pain, such as chronic low back pain, for example with or without previous spine surgery.

Embodiment 13c: A pharmaceutical combination comprising a mGluR5 antagonist, and at least one further pharmaceutical active ingredient, for use according to any one of the preceding embodiments, wherein the mGluR5 antagonist is administered to an elderly patient.

Embodiment 14c: A pharmaceutical combination comprising a mGluR5 antagonist, and at least one further pharmaceutical active ingredient, for use according to any one of the preceding embodiments, wherein chronic pain is chronic post-surgical neuropathic pain.

Embodiment 15c: A pharmaceutical combination comprising a mGluR5 antagonist, and at least one further pharmaceutical active ingredient, for use according to any one of the preceding embodiments, wherein the mGluR5 antagonist is administered in an immediate-release form or a modified-release form; in particular a modified-release form.

Embodiment 16c: A pharmaceutical combination comprising a mGluR5 antagonist, and at least one further pharmaceutical active ingredient, for use according to any one of the preceding embodiments, wherein the mGluR5 antagonist is selected from the group consisting of Compound (I), Compound (II), and Compound (III); or in each instance a pharmaceutically acceptable salt thereof.

Embodiment 17c: A pharmaceutical combination comprising a mGluR5 antagonist, and at least one further pharmaceutical active ingredient, for use according to embodiment 16c, wherein the mGluR5 antagonist is Compound (I) or a pharmaceutically acceptable salt thereof.

Embodiment 18c: A pharmaceutical combination comprising a mGluR5 antagonist, and at least one further pharmaceutical active ingredient, for use according to embodiment 17c, wherein the mGluR5 antagonist is administered in an amount of from 50 mg/b.i.d to 200 mg/b.i.d, in particular 50 mg/b.i.d., 100 mg/b.i.d or 200 mg/b.i.d., such as 200 mg/b.i.d.

Embodiment 19c: A pharmaceutical combination comprising a mGluR5 antagonist, and at least one further pharmaceutical active ingredient, for use according to embodiment 16c, wherein the mGluR5 antagonist is Compound (II) or a pharmaceutically acceptable salt thereof.

Embodiment 20c: A pharmaceutical combination comprising a mGluR5 antagonist, and at least one further pharmaceutical active ingredient, for use according to embodiment 19c, wherein the mGluR5 antagonist is administered in an amount of from 1 mg/day to 100 mg/day.

Embodiment 21c: A pharmaceutical combination comprising a mGluR5 antagonist, and at least one further pharmaceutical active ingredient, for use according to embodiment 16c, wherein the mGluR5 antagonist is Compound (III) or a pharmaceutically acceptable salt thereof.

Embodiments (d):

Embodiment 1d: A method for treating opioid analgesic tolerance associated with chronic opioid use in chronic pain, in a subject in need thereof, comprising administering to said subject an effective amount of a mGluR5 antagonist.

Embodiment 2d: A method for treating opioid analgesic tolerance associated with chronic opioid use in chronic pain, the treatment reversing opioid analgesic tolerance, in a subject in need thereof, comprising administering to said subject an effective amount of a mGluR5 antagonist.

Embodiment 3d: A method of treatment for reversing opioid analgesic tolerance associated with chronic opioid use in chronic pain, in a subject in need thereof, comprising administering to said subject an effective amount of a mGluR5 antagonist.

Embodiment 4d: A method of treatment to reduce the risk of opioid-related overdose associated with chronic opioid use in chronic pain, in a subject in need thereof, comprising administering to said subject an effective amount of a mGluR5 antagonist.

Embodiment 5d: A method of treatment of chronic pain in order to reverse opioid analgesic tolerance associated with chronic opioid use, in a subject in need thereof, comprising administering to said subject an effective amount of a mGluR5 antagonist.

Embodiment 6d: A method of treatment of opioid analgesic tolerance in order to reduce the risk of opioid-related overdose associated with chronic opioid use in chronic pain, in a subject in need thereof, comprising administering to said subject an effective amount of a mGluR5 antagonist.

Embodiment 7d: A method of treatment for reducing opioid consumption in chronic pain, in a subject in need thereof, comprising administering to said subject an effective amount of a mGluR5 antagonist.

Embodiment 8d: A method of treatment for reversing opioid analgesic tolerance associated with chronic opioid use in chronic pain, in a subject in need thereof, comprising administering to said subject an effective amount of a mGluR5 antagonist, whereby the opioid dosage [e.g. daily opioid dose] being administered to the patient is decreased (e.g. by 50% or more, such as by 70% or more, e.g. by 90% or more) and the opioid analgesic effect (i.e. previously achieved by using the higher opioid dosage) is maintained.

Embodiment 9d: A method according to any one of the preceding embodiments, wherein the mGluR5 antagonist is administered to a patient on an opioid dose of >50 mg oral morphine/day or daily equianalgesic dose of another opioid (i.e. morphine milligram equivalent MME/day), such as a patient on an opioid dose of from 60 to 100 mg oral morphine/day or daily equianalgesic dose of another opioid.

Embodiment 10d: A method according to any one of the preceding embodiments, wherein chronic pain is associated to an injury (e.g. wound, burn or fracture) or a disease (e.g. diabetes, multiple sclerosis, arthritis, an autoimmune disease or an infection).

Embodiment 11d: A method according to any one of the preceding embodiments, wherein chronic pain is non-malignant chronic pain.

Embodiment 12d: A method according to embodiment 11d, wherein non-malignant chronic pain is chronic back pain, such as chronic low back pain, for example with or without previous spine surgery.

Embodiment 13d: A method according to any one of the preceding embodiments, wherein the mGluR5 antagonist is administered to an elderly patient.

Embodiment 14d: A method according to any one of the preceding embodiments, wherein chronic pain is chronic post-surgical neuropathic pain.

Embodiment 15d: A method according to any one of the preceding embodiments, wherein the mGluR5 antagonist is administered in an immediate-release form or a modified-release form; in particular a modified-release form.

Embodiment 16d: A method according to any one of the preceding embodiments, wherein the mGluR5 antagonist is administered in the form of a pharmaceutical composition further comprising at least one pharmaceutically acceptable excipient.

Embodiment 17d: A method according to any one of the preceding embodiments, wherein the mGluR5 antagonist is administered in combination with one or more further pharmaceutical active ingredient.

Embodiment 18d: A method according to any one of the preceding embodiments, wherein the mGluR5 antagonist is selected from the group consisting of Compound (I), Compound (II), and Compound (III); or in each instance a pharmaceutically acceptable salt thereof.

Embodiment 19d: A method according to embodiment 18d, wherein the mGluR5 antagonist is Compound (I) or a pharmaceutically acceptable salt thereof.

Embodiment 20d: A method according to embodiment 19d, wherein the mGluR5 antagonist is administered in an amount of from 50 mg/b.i.d to 200 mg/b.i.d, in particular 50 mg/b.i.d., 100 mg/b.i.d or 200 mg/b.i.d., such as 200 mg/b.i.d.

Embodiment 21d: A method according to embodiment 18d, wherein the mGluR5 antagonist is Compound (II) or a pharmaceutically acceptable salt thereof.

Embodiment 22d: A method according to embodiment 21d, wherein the mGluR5 antagonist is administered in an amount of from 1 mg/day to 100 mg/day.

Embodiment 23d: A method according to embodiment 18d, wherein the mGluR5 antagonist is Compound (III) or a pharmaceutically acceptable salt thereof.

Embodiments (e):

Embodiment 1e: A method for treating opioid analgesic tolerance associated with chronic opioid use in chronic pain, in a subject in need thereof, comprising administering to said subject a pharmaceutical composition comprising an effective amount of a mGluR5 antagonist and at least one pharmaceutically acceptable excipient.

Embodiment 2e: A method for treating opioid analgesic tolerance associated with chronic opioid use in chronic pain, the treatment reversing opioid analgesic tolerance, in a subject in need thereof, comprising administering to said subject a pharmaceutical composition comprising an effective amount of a mGluR5 antagonist and at least one pharmaceutically acceptable excipient.

Embodiment 3e: A method of treatment for reversing opioid analgesic tolerance associated with chronic opioid use in chronic pain, in a subject in need thereof, comprising administering to said subject a pharmaceutical composition comprising an effective amount of a mGluR5 antagonist and at least one pharmaceutically acceptable excipient.

Embodiment 4e: A method of treatment to reduce the risk of opioid-related overdose associated with chronic opioid use in chronic pain, in a subject in need thereof, comprising administering to said subject a pharmaceutical composition comprising an effective amount of a mGluR5 antagonist and at least one pharmaceutically acceptable excipient.

Embodiment 5e: A method of treatment of chronic pain in order to reverse opioid analgesic tolerance associated with chronic opioid use, in a subject in need thereof, comprising administering to said subject a pharmaceutical composition comprising an effective amount of a mGluR5 antagonist and at least one pharmaceutically acceptable excipient.

Embodiment 6e: A method of treatment of opioid analgesic tolerance in order to reduce the risk of opioid-related overdose associated with chronic opioid use in chronic pain, in a subject in need thereof, comprising administering to said subject a pharmaceutical composition comprising an effective amount of a mGluR5 antagonist and at least one pharmaceutically acceptable excipient.

Embodiment 7e: A method of treatment for reducing opioid consumption in chronic pain, in a subject in need thereof, comprising administering to said subject a pharmaceutical composition comprising an effective amount of a mGluR5 antagonist and at least one pharmaceutically acceptable excipient.

Embodiment 8e: A method of treatment for reversing opioid analgesic tolerance associated with chronic opioid use in chronic pain, in a subject in need thereof, comprising administering to said subject a pharmaceutical composition comprising an effective amount of a mGluR5 antagonist and at least one pharmaceutically acceptable excipient, whereby the opioid dosage [e.g. daily opioid dose] being administered to the patient is decreased (e.g. by 50% or more, such as by 70% or more, e.g. by 90% or more) and the opioid analgesic effect (i.e. previously achieved by using the higher opioid dosage) is maintained.

Embodiment 9e: A method according to any one of the preceding embodiments, wherein the mGluR5 antagonist is administered to a patient on an opioid dose of >50 mg oral morphine/day or daily equianalgesic dose of another opioid (i.e. morphine milligram equivalent MME/day), such as a patient on an opioid dose of from 60 to 100 mg oral morphine/day or daily equianalgesic dose of another opioid.

Embodiment 10e: A method according to any one of the preceding embodiments, wherein chronic pain is associated to an injury (e.g. wound, burn or fracture) or a disease (e.g. diabetes, multiple sclerosis, arthritis, an autoimmune disease or an infection).

Embodiment 11e: A method according to any one of the preceding embodiments, wherein chronic pain is non-malignant chronic pain.

Embodiment 12e: A method according to embodiment 11e, wherein non-malignant chronic pain is chronic back pain, such as chronic low back pain, for example with or without previous spine surgery.

Embodiment 13e: A mGluR5 antagonist for use according to any one of the preceding embodiments, wherein the mGluR5 antagonist is administered to an elderly patient.

Embodiment 14e: A method according to any one of the preceding embodiments, wherein chronic pain is chronic post-surgical neuropathic pain.

Embodiment 15e: A method according to any one of the preceding embodiments, wherein the mGluR5 antagonist is administered in an immediate-release form or a modified-release form; in particular a modified-release form.

Embodiment 16e: A method according to any one of the preceding embodiments, wherein the mGluR5 antagonist is administered in combination with one or more further pharmaceutical active ingredient.

Embodiment 17e: A method according to any one of the preceding embodiments, wherein the mGluR5 antagonist is selected from the group consisting of Compound (I), Compound (II), and Compound (III); or in each instance a pharmaceutically acceptable salt thereof.

Embodiment 18e: A method according to embodiment 17e, wherein the mGluR5 antagonist is Compound (I) or a pharmaceutically acceptable salt thereof.

Embodiment 19e: A method according to embodiment 18e, wherein the mGluR5 antagonist is administered in an amount of from 50 mg/b.i.d to 200 mg/b.i.d, in particular 50 mg/b.i.d., 100 mg/b.i.d or 200 mg/b.i.d., such as 200 mg/b.i.d.

Embodiment 20e: A method according to embodiment 17e, wherein the mGluR5 antagonist is Compound (II) or a pharmaceutically acceptable salt thereof.

Embodiment 21e: A method according to embodiment 20e, wherein the mGluR5 antagonist is administered in an amount of from 1 mg/day to 100 mg/day.

Embodiment 22e: A method according to embodiment 17e, wherein the mGluR5 antagonist is Compound (III) or a pharmaceutically acceptable salt thereof.

Embodiments (f):

Embodiment 1f: A method for treating opioid analgesic tolerance associated with chronic opioid use in chronic pain, in a subject in need thereof, comprising administering to said subject a pharmaceutical combination comprising an effective amount of a mGluR5 antagonist and at least one further pharmaceutical active ingredient.

Embodiment 2f: A method for treating opioid analgesic tolerance associated with chronic opioid use in chronic pain, the treatment reversing opioid analgesic tolerance, in a subject in need thereof, comprising administering to said subject a pharmaceutical combination comprising an effective amount of a mGluR5 antagonist and at least one further pharmaceutical active ingredient.

Embodiment 3f: A method of treatment for reversing opioid analgesic tolerance associated with chronic opioid use in chronic pain, in a subject in need thereof, comprising administering to said subject a pharmaceutical combination comprising an effective amount of a mGluR5 antagonist and at least one further pharmaceutical active ingredient.

Embodiment 4f: A method of treatment to reduce the risk of opioid-related overdose associated with chronic opioid use in chronic pain, in a subject in need thereof, comprising administering to said subject a pharmaceutical combination comprising an effective amount of a mGluR5 antagonist and at least one further pharmaceutical active ingredient.

Embodiment 5f: A method of treatment of chronic pain in order to reverse opioid analgesic tolerance associated with chronic opioid use, in a subject in need thereof, comprising administering to said subject a pharmaceutical combination comprising an effective amount of a mGluR5 antagonist and at least one further pharmaceutical active ingredient.

Embodiment 6f: A method of treatment of opioid analgesic tolerance in order to reduce the risk of opioid-related overdose associated with chronic opioid use in chronic pain, in a subject in need thereof, comprising administering to said subject a pharmaceutical combination comprising an effective amount of a mGluR5 antagonist and at least one further pharmaceutical active ingredient.

Embodiment 7f: A method of treatment for reducing opioid consumption in chronic pain, in a subject in need thereof, comprising administering to said subject a pharmaceutical combination comprising an effective amount of a mGluR5 antagonist and at least one further pharmaceutical active ingredient.

Embodiment 8f: A method of treatment for reversing opioid analgesic tolerance associated with chronic opioid use in chronic pain, in a subject in need thereof, comprising administering to said subject a pharmaceutical combination comprising an effective amount of a mGluR5 antagonist and at least one further pharmaceutical active ingredient, whereby the opioid dosage [e.g. daily opioid dose] being administered to the patient is decreased (e.g. by 50% or more, such as by 70% or more, e.g. by 90% or more) and the opioid analgesic effect (i.e. previously achieved by using the higher opioid dosage) is maintained.

Embodiment 9f: A method according to any one of the preceding embodiments, wherein the mGluR5 antagonist is administered to a patient on an opioid dose of >50 mg oral morphine/day or daily equianalgesic dose of another opioid (i.e. morphine milligram equivalent MME/day), such as a patient on an opioid dose of from 60 to 100 mg oral morphine/day or daily equianalgesic dose of another opioid.

Embodiment 10f: A method according to any one of the preceding embodiments, wherein chronic pain is associated to an injury (e.g. wound, burn or fracture) or a disease (e.g. diabetes, multiple sclerosis, arthritis, an autoimmune disease or an infection).

Embodiment 11f: A method according to any one of the preceding embodiments, wherein chronic pain is non-malignant chronic pain.

Embodiment 12f: A method according to embodiment 11f, wherein non-malignant chronic pain is chronic back pain, such as chronic low back pain, for example with or without previous spine surgery.

Embodiment 13f: A mGluR5 antagonist for use according to any one of the preceding embodiments, wherein the mGluR5 antagonist is administered to an elderly patient.

Embodiment 14f: A method according to any one of the preceding embodiments, wherein chronic pain is chronic post-surgical neuropathic pain.

Embodiment 15f: A method according to any one of the preceding embodiments, wherein the mGluR5 antagonist is administered in an immediate-release form or a modified-release form; in particular a modified-release form.

Embodiment 16f: A method according to any one of the preceding embodiments, wherein the mGluR5 antagonist is selected from the group consisting of Compound (I), Compound (II), and Compound (III); or in each instance a pharmaceutically acceptable salt thereof.

Embodiment 17f: A method according to embodiment 16f, wherein the mGluR5 antagonist is Compound (I) or a pharmaceutically acceptable salt thereof.

Embodiment 18f: A method according to embodiment 17f, wherein the mGluR5 antagonist is administered in an amount of from 50 mg/b.i.d to 200 mg/b.i.d, in particular 50 mg/b.i.d., 100 mg/b.i.d or 200 mg/b.i.d., such as 200 mg/b.i.d.

Embodiment 19f: A method according to embodiment 16f, wherein the mGluR5 antagonist is Compound (II) or a pharmaceutically acceptable salt thereof.

Embodiment 20f: A method according to embodiment 19f, wherein the mGluR5 antagonist is administered in an amount of from 1 mg/day to 100 mg/day.

Embodiment 21f: A method according to embodiment 16f, wherein the mGluR5 antagonist is Compound (III) or a pharmaceutically acceptable salt thereof.

Embodiments (g):

Embodiment 1g: Use of a mGluR5 antagonist for the manufacture of a medicament for the treatment of opioid analgesic tolerance associated with chronic opioid use in chronic pain.

Embodiment 2g: Use of a mGluR5 antagonist for the manufacture of a medicament for the treatment of opioid analgesic tolerance associated with chronic opioid use in chronic pain, the treatment reversing opioid analgesic tolerance.

Embodiment 3g: Use of a mGluR5 antagonist for the manufacture of a medicament for a treatment to reverse opioid analgesic tolerance associated with chronic opioid use in chronic pain.

Embodiment 4g: Use of a mGluR5 antagonist for the manufacture of a medicament for a treatment to reduce the risk of opioid-related overdose associated with chronic opioid use in chronic pain.

Embodiment 5g: Use of a mGluR5 antagonist for the manufacture of a medicament for the treatment of chronic pain in order to reverse opioid analgesic tolerance associated with chronic opioid use.

Embodiment 6g: Use of a mGluR5 antagonist for the manufacture of a medicament for the treatment of opioid analgesic tolerance in order to reduce the risk of opioid-related overdose associated with chronic opioid use in chronic pain.

Embodiment 7g: Use of a mGluR5 antagonist for the manufacture of a medicament for a treatment to reduce opioid consumption in chronic pain.

Embodiment 8g: Use of a mGluR5 antagonist for the manufacture of a medicament for a treatment to reverse opioid analgesic tolerance associated with chronic opioid use in chronic pain, wherein the opioid dosage [e.g. daily opioid dose] being administered to the patient is decreased (e.g. by 50% or more, such as by 70% or more, e.g. by 90% or more) and the opioid analgesic effect (i.e. previously achieved by using the higher opioid dosage) is maintained.

Embodiment 9g: Use of a mGluR5 antagonist for the manufacture of a medicament according to any one of the preceding embodiments, wherein the mGluR5 antagonist is administered to a patient on an opioid dose of >50 mg oral morphine/day or daily equianalgesic dose of another opioid (i.e. morphine milligram equivalent MME/day), such as a patient on an opioid dose of from 60 to 100 mg oral morphine/day or daily equianalgesic dose of another opioid.

Embodiment 10g: Use of a mGluR5 antagonist for the manufacture of a medicament according to any one of the preceding embodiments, wherein chronic pain is associated to an injury (e.g. wound, burn or fracture) or a disease (e.g. diabetes, multiple sclerosis, arthritis, an autoimmune disease or an infection).

Embodiment 11g: Use of a mGluR5 antagonist for the manufacture of a medicament according to any one of the preceding embodiments, wherein chronic pain is non-malignant chronic pain.

Embodiment 12g: Use of a mGluR5 antagonist for the manufacture of a medicament according to embodiment 11g, wherein non-malignant chronic pain is chronic back pain, such as chronic low back pain, for example with or without previous spine surgery.

Embodiment 13g: Use of a mGluR5 antagonist for the manufacture of a medicament according to any one of the preceding embodiments, wherein the mGluR5 antagonist is administered to an elderly patient.

Embodiment 14g: Use of a mGluR5 antagonist for the manufacture of a medicament according to any one of the preceding embodiments, wherein chronic pain is chronic post-surgical neuropathic pain.

Embodiment 15g: Use of a mGluR5 antagonist for the manufacture of a medicament according to any one of the preceding embodiments, wherein the mGluR5 antagonist is administered in an immediate-release form or a modified-release form; in particular a modified-release form.

Embodiment 16g: Use of a mGluR5 antagonist for the manufacture of a medicament according to any one of the preceding embodiments, wherein the mGluR5 antagonist is administered in the form of a pharmaceutical composition further comprising at least one pharmaceutically acceptable excipient.

Embodiment 17g: Use of a mGluR5 antagonist for the manufacture of a medicament according to any one of the preceding embodiments, wherein the mGluR5 antagonist is administered in combination with one or more further pharmaceutical active ingredient.

Embodiment 18g: Use of a mGluR5 antagonist for the manufacture of a medicament according to any one of the preceding embodiments, wherein the mGluR5 antagonist is selected from the group consisting of Compound (I), Compound (II), and Compound (III); or in each instance a pharmaceutically acceptable salt thereof.

Embodiment 19g: Use of a mGluR5 antagonist for the manufacture of a medicament according to embodiment 18g, wherein the mGluR5 antagonist is Compound (I) or a pharmaceutically acceptable salt thereof.

Embodiment 20g: Use of a mGluR5 antagonist for the manufacture of a medicament according to embodiment 19g, wherein the mGluR5 antagonist is administered in an amount of from 50 mg/b.i.d to 200 mg/b.i.d, in particular 50 mg/b.i.d., 100 mg/b.i.d or 200 mg/b.i.d., such as 200 mg/b.i.d.

Embodiment 21g: Use of a mGluR5 antagonist for the manufacture of a medicament according to embodiment 18g, wherein the mGluR5 antagonist is Compound (II) or a pharmaceutically acceptable salt thereof.

Embodiment 22g: Use of a mGluR5 antagonist for the manufacture of a medicament according to embodiment 21g, wherein the mGluR5 antagonist is administered in an amount of from 1 mg/day to 100 mg/day.

Embodiment 23g: Use of a mGluR5 antagonist for the manufacture of a medicament according to embodiment 18g, wherein the mGluR5 antagonist is Compound (III) or a pharmaceutically acceptable salt thereof.

Embodiments (h):

Embodiment 1h: Use of a pharmaceutical composition comprising a mGluR5 antagonist, and at least one pharmaceutically acceptable excipient, for the manufacture of a medicament for the treatment of opioid analgesic tolerance associated with chronic opioid use in chronic pain.

Embodiment 2h: Use of a pharmaceutical composition comprising a mGluR5 antagonist, and at least one pharmaceutically acceptable excipient, for the manufacture of a medicament for the treatment of opioid analgesic tolerance associated with chronic opioid use in chronic pain, the treatment reversing opioid analgesic tolerance.

Embodiment 3h: Use of a pharmaceutical composition comprising a mGluR5 antagonist, and at least one pharmaceutically acceptable excipient, for the manufacture of a medicament for a treatment to reverse opioid analgesic tolerance associated with chronic opioid use in chronic pain.

Embodiment 4h: Use of a pharmaceutical composition comprising a mGluR5 antagonist, and at least one pharmaceutically acceptable excipient, for the manufacture of a medicament for a treatment to reduce the risk of opioid-related overdose associated with chronic opioid use in chronic pain.

Embodiment 5h: Use of a pharmaceutical composition comprising a mGluR5 antagonist, and at least one pharmaceutically acceptable excipient, for the manufacture of a medicament for the treatment of chronic pain in order to reverse opioid analgesic tolerance associated with chronic opioid use.

Embodiment 6h: Use of a pharmaceutical composition comprising a mGluR5 antagonist, and at least one pharmaceutically acceptable excipient, for the manufacture of a medicament for the treatment of opioid analgesic tolerance in order to reduce the risk of opioid-related overdose associated with chronic opioid use in chronic pain.

Embodiment 7h: Use of a pharmaceutical composition comprising a mGluR5 antagonist, and at least one pharmaceutically acceptable excipient, for the manufacture of a medicament for a treatment to reduce opioid consumption in chronic pain.

Embodiment 8h: Use of a pharmaceutical composition comprising a mGluR5 antagonist, and at least one pharmaceutically acceptable excipient, for the manufacture of a medicament for a treatment to reverse opioid analgesic tolerance associated with chronic opioid use in chronic pain, wherein the opioid dosage [e.g. daily opioid dose] being administered to the patient is decreased (e.g. by 50% or more, such as by 70% or more, e.g. by 90% or more) and the opioid analgesic effect (i.e. previously achieved by using the higher opioid dosage) is maintained.

Embodiment 9h: Use of a pharmaceutical composition comprising, and at least one pharmaceutically acceptable excipient, a mGluR5 antagonist for the manufacture of a medicament according to any one of the preceding embodiments, wherein the mGluR5 antagonist is administered to a patient on an opioid dose of >50 mg oral morphine/day or daily equianalgesic dose of another opioid (i.e. morphine milligram equivalent MME/day), such as a patient on an opioid dose of from 60 to 100 mg oral morphine/day or daily equianalgesic dose of another opioid.

Embodiment 10h: Use of a pharmaceutical composition comprising a mGluR5 antagonist, and at least one pharmaceutically acceptable excipient, for the manufacture of a medicament according to any one of the preceding embodiments, wherein chronic pain is associated to an injury (e.g. wound, burn or fracture) or a disease (e.g. diabetes, multiple sclerosis, arthritis, an autoimmune disease or an infection).

Embodiment 11h: Use of a pharmaceutical composition comprising a mGluR5 antagonist, and at least one pharmaceutically acceptable excipient, for the manufacture of a medicament according to any one of the preceding embodiments, wherein chronic pain is non-malignant chronic pain.

Embodiment 12h: Use of a pharmaceutical composition comprising a mGluR5 antagonist, and at least one pharmaceutically acceptable excipient, for the manufacture of a medicament according to embodiment 11h, wherein non-malignant chronic pain is chronic back pain, such as chronic low back pain, for example with or without previous spine surgery.

Embodiment 13h: Use of a pharmaceutical composition comprising a mGluR5 antagonist, and at least one pharmaceutically acceptable excipient, for the manufacture of a medicament according to any one of the preceding embodiments, wherein the mGluR5 antagonist is administered to an elderly patient.

Embodiment 14h: Use of a pharmaceutical composition comprising a mGluR5 antagonist, and at least one pharmaceutically acceptable excipient, for the manufacture of a medicament according to any one of the preceding embodiments, wherein chronic pain is chronic post-surgical neuropathic pain.

Embodiment 15h: Use of a pharmaceutical composition comprising a mGluR5 antagonist, and at least one pharmaceutically acceptable excipient, for the manufacture of a medicament according to any one of the preceding embodiments, wherein the mGluR5 antagonist is administered in an immediate-release form or a modified-release form; in particular a modified-release form.

Embodiment 16h: Use of a pharmaceutical composition comprising a mGluR5 antagonist, and at least one pharmaceutically acceptable excipient, for the manufacture of a medicament according to any one of the preceding embodiments, wherein the mGluR5 antagonist is administered in combination with one or more further pharmaceutical active ingredient.

Embodiment 17h: Use of a pharmaceutical composition comprising a mGluR5 antagonist, and at least one pharmaceutically acceptable excipient, for the manufacture of a medicament according to any one of the preceding embodiments, wherein the mGluR5 antagonist is selected from the group consisting of Compound (I), Compound (II), and Compound (III); or in each instance a pharmaceutically acceptable salt thereof.

Embodiment 18h: Use of a pharmaceutical composition comprising a mGluR5 antagonist, and at least one pharmaceutically acceptable excipient, for the manufacture of a medicament according to embodiment 17h, wherein the mGluR5 antagonist is Compound (I) or a pharmaceutically acceptable salt thereof.

Embodiment 19h: Use of a pharmaceutical composition comprising a mGluR5 antagonist, and at least one pharmaceutically acceptable excipient, for the manufacture of a medicament according to embodiment 18h, wherein the mGluR5 antagonist is administered in an amount of from 50 mg/b.i.d to 200 mg/b.i.d, in particular 50 mg/b.i.d., 100 mg/b.i.d or 200 mg/b.i.d., such as 200 mg/b.i.d.

Embodiment 20h: Use of a pharmaceutical composition comprising a mGluR5 antagonist, and at least one pharmaceutically acceptable excipient, for the manufacture of a medicament according to embodiment 17h, wherein the mGluR5 antagonist is Compound (II) or a pharmaceutically acceptable salt thereof.

Embodiment 21h: Use of a pharmaceutical composition comprising a mGluR5 antagonist, and at least one pharmaceutically acceptable excipient, for the manufacture of a medicament according to embodiment 20h, wherein the mGluR5 antagonist is administered in an amount of from 1 mg/day to 100 mg/day.

Embodiment 22h: Use of a pharmaceutical composition comprising a mGluR5 antagonist, and at least one pharmaceutically acceptable excipient, for the manufacture of a medicament according embodiment 17h, wherein the mGluR5 antagonist is Compound (III) or a pharmaceutically acceptable salt thereof.

Embodiments (j):

Embodiment 1j: Use of a pharmaceutical combination comprising a mGluR5 antagonist, and at least one further pharmaceutical active ingredient, for the manufacture of a medicament for the treatment of opioid analgesic tolerance associated with chronic opioid use in chronic pain.

Embodiment 2j: Use of a pharmaceutical combination comprising a mGluR5 antagonist, and at least one further pharmaceutical active ingredient, for the manufacture of a medicament for the treatment of opioid analgesic tolerance associated with chronic opioid use in chronic pain, the treatment reversing opioid analgesic tolerance.

Embodiment 3j: Use of a pharmaceutical combination comprising a mGluR5 antagonist, and at least one further pharmaceutical active ingredient, for the manufacture of a medicament for a treatment to reverse opioid analgesic tolerance associated with chronic opioid use in chronic pain.

Embodiment 4j: Use of a pharmaceutical combination comprising a mGluR5 antagonist, and at least one further pharmaceutical active ingredient, for the manufacture of a medicament for a treatment to reduce the risk of opioid-related overdose associated with chronic opioid use in chronic pain.

Embodiment 5j: Use of a pharmaceutical combination comprising a mGluR5 antagonist, and at least one further pharmaceutical active ingredient, for the manufacture of a medicament for the treatment of chronic pain in order to reverse opioid analgesic tolerance associated with chronic opioid use.

Embodiment 6j: Use of a pharmaceutical combination comprising a mGluR5 antagonist, and at least one further pharmaceutical active ingredient, for the manufacture of a medicament for the treatment of opioid analgesic tolerance in order to reduce the risk of opioid-related overdose associated with chronic opioid use in chronic pain.

Embodiment 7j: Use of a pharmaceutical combination comprising a mGluR5 antagonist, and at least one further pharmaceutical active ingredient, for the manufacture of a medicament for a treatment to reduce opioid consumption in chronic pain.

Embodiment 8j: Use of a pharmaceutical combination comprising a mGluR5 antagonist, and at least one further pharmaceutical active ingredient, for the manufacture of a medicament for a treatment to reverse opioid analgesic tolerance associated with chronic opioid use in chronic pain, wherein the opioid dosage [e.g. daily opioid dose] being administered to the patient is decreased (e.g. by 50% or more, such as by 70% or more, e.g. by 90% or more) and the opioid analgesic effect (i.e. previously achieved by using the higher opioid dosage) is maintained.

Embodiment 9j: Use of a pharmaceutical combination comprising, and at least one further pharmaceutical active ingredient, a mGluR5 antagonist for the manufacture of a medicament according to any one of the preceding embodiments, wherein the mGluR5 antagonist is administered to a patient on an opioid dose of >50 mg oral morphine/day or daily equianalgesic dose of another opioid (i.e. morphine milligram equivalent MME/day), such as a patient on an opioid dose of from 60 to 100 mg oral morphine/day or daily equianalgesic dose of another opioid.

Embodiment 10j: Use of a pharmaceutical combination comprising a mGluR5 antagonist, and at least one further pharmaceutical active ingredient, for the manufacture of a medicament according to any one of the preceding embodiments, wherein chronic pain is associated to an injury (e.g. wound, burn or fracture) or a disease (e.g. diabetes, multiple sclerosis, arthritis, an autoimmune disease or an infection).

Embodiment 11j: Use of a pharmaceutical combination comprising a mGluR5 antagonist, and at least one further pharmaceutical active ingredient, for the manufacture of a medicament according to any one of the preceding embodiments, wherein chronic pain is non-malignant chronic pain.

Embodiment 12j: Use of a pharmaceutical combination comprising a mGluR5 antagonist, and at least one further pharmaceutical active ingredient, for the manufacture of a medicament according to embodiment 11j, wherein non-malignant chronic pain is chronic back pain, such as chronic low back pain, for example with or without previous spine surgery.

Embodiment 13j: Use of a pharmaceutical combination comprising a mGluR5 antagonist, and at least one further pharmaceutical active ingredient, for the manufacture of a medicament according to any one of the preceding embodiments, wherein the mGluR5 antagonist is administered to an elderly patient.

Embodiment 14j: Use of a pharmaceutical combination comprising a mGluR5 antagonist, and at least one further pharmaceutical active ingredient, for the manufacture of a medicament according to any one of the preceding embodiments, wherein chronic pain is chronic post-surgical neuropathic pain.

Embodiment 15j: Use of a pharmaceutical combination comprising a mGluR5 antagonist, and at least one further pharmaceutical active ingredient, for the manufacture of a medicament according to any one of the preceding embodiments, wherein the mGluR5 antagonist is administered in an immediate-release form or a modified-release form; in particular a modified-release form.

Embodiment 16j: Use of a pharmaceutical combination comprising a mGluR5 antagonist, and at least one further pharmaceutical active ingredient, for the manufacture of a medicament according to any one of the preceding embodiments, wherein the mGluR5 antagonist is selected from the group consisting of Compound (I), Compound (II), and Compound (III); or in each instance a pharmaceutically acceptable salt thereof.

Embodiment 17j: Use of a pharmaceutical combination comprising a mGluR5 antagonist, and at least one further pharmaceutical active ingredient, for the manufacture of a medicament according to embodiment 16j, wherein the mGluR5 antagonist is Compound (I) or a pharmaceutically acceptable salt thereof.

Embodiment 18j: Use of a pharmaceutical combination comprising a mGluR5 antagonist, and at least one further pharmaceutical active ingredient, for the manufacture of a medicament according to embodiment 17j, wherein the mGluR5 antagonist is administered in an amount of from 50 mg/b.i.d to 200 mg/b.i.d, in particular 50 mg/b.i.d., 100 mg/b.i.d or 200 mg/b.i.d., such as 200 mg/b.i.d.

Embodiment 19j: Use of a pharmaceutical combination comprising a mGluR5 antagonist, and at least one further pharmaceutical active ingredient, for the manufacture of a medicament according to embodiment 16j, wherein the mGluR5 antagonist is Compound (II) or a pharmaceutically acceptable salt thereof.

Embodiment 20j: Use of a pharmaceutical combination comprising a mGluR5 antagonist, and at least one further pharmaceutical active ingredient, for the manufacture of a medicament according to embodiment 19j, wherein the mGluR5 antagonist is administered in an amount of from 1 mg/day to 100 mg/day.

Embodiment 21j: Use of a pharmaceutical combination comprising a mGluR5 antagonist, and at least one further pharmaceutical active ingredient, for the manufacture of a medicament according to embodiment 16j, wherein the mGluR5 antagonist is Compound (III) or a pharmaceutically acceptable salt thereof.

### General Terms

The term “opioid” or “opioid analgesic”, as used herein, refers to both natural (opiate) or synthetic (opioids) forms, as well as natural or synthetic mixed opioid agonists/antagonists, and include, without limitation, alphamethylfentanyl, alfentanil, buprenorphine, butorphanol, codeine, diacetylmorphine, dihydrocodeine, dihydroetorphine, dihydromorphine, ethylmorphine, etorphine, fentanyl, hydrocodone, hydromorphone, L-acetylmethadol, levorphanol, methadone, meperidine, morphine, nicomorphine, normethadone, noroxycodone, normorphine, norlevorphanol, oxycodone, oxymorphone, phenazocine, propoxyphene, remifentanil, tramadol, thebaine, tapentadol, levorphanol, sufentanil, pentazocine, carfentanyl, ohmfentanyl, nocaine, ketobemidone, allylprodine, prodine, dextropropoxyphene, dextromoramide, benzitramide, piritramide, dipipanone, loperamide, diphenoxylate, nalbuphine, levomethorpha, dezocine, lefetamine and tildine. In one embodiment, “opioid” is selected from the group consisting of morphine, hydrocodone, oxycodone and codeine; in particular the opioid is selected from the group consisting of hydrocodone and oxycodone. In one embodiment, the opioid is hydrocodone. In another embodiment, the opioid is oxycodone.
The term “chronic opioid use” or “chronic use of an opioid”, as used herein, refers to opioid treatment/therapy (e.g. daily) lasting more than 6 months, wherein the opioid is, for example, as defined herein. In one embodiment “chronic opioid use” refers to chronic alphamethylfentanyl use, chronic alfentanil use, chronic buprenorphine use, chronic butorphanol use, chronic codeine use, chronic diacetylmorphine use, chronic dihydrocodeine use, chronic dihydroetorphine use, chronic dihydromorphine use, chronic ethylmorphine use, chronic etorphine use, chronic fentanyl analgesic use, chronic hydrocodone use, chronic hydromorphone use, chronic L-acetylmethadol use, chronic levorphanol use, chronic methadone use, chronic meperidine use, chronic morphine use, chronic nicomorphine use, chronic normethadone use, chronic noroxycodone use, chronic normorphine use, chronic norlevorphanol use, chronic oxycodone use, chronic oxymorphone use, chronic phenazocine use, chronic propoxyphene use, chronic remifentanil use, chronic tramadol use, chronic thebaine use, chronic tapentadol use, chronic levorphanol use, chronic sufentanil use, chronic pentazocine use, chronic carfentanyl use, chronic ohmfentanyl use, chronic nocaine use, chronic ketobemidone use, chronic allylprodine use, chronic prodine use, chronic dextropropoxyphene use, chronic dextromoramide use, chronic benzitramide use, chronic piritramide use, chronic dipipanone use, chronic loperamide use, chronic diphenoxylate use, chronic nalbuphine use, chronic levomethorpha use, chronic dezocine use, chronic lefetamine use and chronic tildine use; wherein the term “chronic” in each instance refers to use (e.g. daily) lasting more than 6 months. In another embodiment “chronic opioid use” refers to chronic morphine use, chronic hydrocodone use, chronic oxycodone use or chronic codeine use, in one embodiment, chronic hydrocodone use, in another embodiment chronic oxycodone use; wherein the term “chronic” in each instance refers to use (e.g. daily) lasting more than 6 months.
The term “chronic opioid use in chronic pain”, as used herein, is to be understood as “chronic opioid use”, as defined herein, by a patient with chronic pain. The term “chronic opioid user” refers to a chronic pain patient on opioids (e.g. daily), as defined herein, lasting more than 6 months.
The term “opioid treatment” or “opioid therapy”, as used herein, refers to analgesic treatment of chronic pain with an opioid, for example as defined herein [see, for example, the Interagency Guideline on Prescribing Opioids for Pain, 3^{rd} Edition, June 2015, or the Centers for Disease Control and Prevention (CDC) Guideline for Prescribing Opioids for Chronic Pain, 2016, or in The Journal of Pain, 2009, 10(2), 113-130; the entire contents of which are incorporated herein by reference].
The term “tolerance”, as used herein, is understood, for example, according to the definition by the National Institute on Drug Abuse (NIDA), namely a state in which the patient, no longer responds to a drug and a higher dose (e.g. higher daily dose) is required to achieve the effect. Tolerance is thus a phenomenon of the body, over a period of time, in which one or more effects of the drug diminish with repeated use at the same dose and a higher dose is required to achieve results (e.g. need of increased dosage to maintain analgesic effect).

The term “opioid tolerance” or “opioid analgesic tolerance”, as used herein, refers to the chronic use of an opioid which leads to a progressively dose increase (e.g. daily dose increase) of the opioid in order to maintain analgesic effect. Evidence of tolerance may be assessed, for example, according to the PEG scale [i.e. assessment of pain intensity (P), enjoyment of life (E), general activity (G); the PEG scale has shown to be sensitive to change and differentiation between patients with and without pain improvement; e.g. in http://mytopcare.org/wp-content/uploads/2013/06/PEG-Pain-Screening-Tool1.pdf, Krebs E E, et. al. in J. Gen. Intern. Med, 2009, 24, 733-8]. In one embodiment, “opioid analgesic tolerance” is selected from the group consisting of alphamethylfentanyl analgesic tolerance, alfentanil analgesic tolerance, buprenorphine analgesic tolerance, butorphanol analgesic tolerance, codeine analgesic tolerance, diacetylmorphine analgesic tolerance, dihydrocodeine analgesic tolerance, dihydroetorphine analgesic tolerance, dihydromorphine analgesic tolerance, ethylmorphine analgesic tolerance, etorphine analgesic tolerance, fentanyl analgesic tolerance, hydrocodone analgesic tolerance, hydromorphone analgesic tolerance, L-acetylmethadol analgesic tolerance, levorphanol analgesic tolerance, methadone analgesic tolerance, meperidine analgesic tolerance, morphine analgesic tolerance, nicomorphine analgesic tolerance, normethadone analgesic tolerance, noroxycodone analgesic, normorphine analgesic tolerance, norlevorphanol analgesic tolerance, oxycodone analgesic tolerance, oxymorphone analgesic tolerance, phenazocine analgesic tolerance, propoxyphene analgesic tolerance, remifentanil analgesic tolerance, tramadol analgesic tolerance associated, thebaine analgesic tolerance, tapentadol analgesic tolerance, levorphanol analgesic tolerance, sufentanil analgesic tolerance, pentazocine analgesic tolerance, carfentanyl analgesic tolerance, ohmfentanyl analgesic tolerance, nocaine analgesic tolerance, ketobemidone analgesic tolerance, allylprodine analgesic tolerance, prodine analgesic tolerance, dextropropoxyphene analgesic tolerance, dextromoramide analgesic tolerance, benzitramide analgesic tolerance, piritramide analgesic tolerance, dipipanone analgesic tolerance, loperamide analgesic tolerance, diphenoxylate analgesic tolerance, nalbuphine analgesic tolerance, levomethorpha analgesic tolerance, dezocine analgesic tolerance, lefetamine analgesic tolerance and tildine analgesic tolerance. In another embodiment, “opioid analgesic tolerance” is selected from the group consisting of morphine analgesic tolerance, hydrocodone analgesic tolerance, oxycodone analgesic tolerance and codeine analgesic tolerance; in particular selected from the group consisting of hydrocodone analgesic tolerance and oxycodone analgesic tolerance; in one embodiment hydrocodone analgesic tolerance; in another embodiment oxycodone analgesic tolerance.
The term “to reverse opioid analgesic tolerance associated with chronic opioid use in chronic pain”, “reverses opioid analgesic tolerance associated with chronic opioid use in chronic pain”, or “reversing opioid analgesic tolerance associated with chronic opioid use in chronic pain”, as used herein, is to be understood as reversal of existing (i.e. established) opioid analgesic tolerance, as defined herein, in a chronic pain patient, who is a chronic opioid user, as defined herein. In particular, it refers to the ability of the active ingredient, such as a mGluR5 antagonist, for example as defined herein, to reduce the opioid dosage [e.g. the daily opioid dose, such as >50 mg oral morphine/day or daily equianalgesic dose of another opioid (i.e. morphine milligram equivalent MME/day)] of the opioid being administered to the chronic pain patient [e.g. a decrease of 50% or more, of the original opioid consumption per day or week, such as a decrease of 70% or more, for example a decrease of 90% or more; for example, as assessed by using biomarkers for metabolites of opioids or as assessed by using patient's self-reported opioid intake (e.g. by using the Timeline Follow Back Opioid Self-reported Diary)] and maintain analgesic effect (i.e. maintain analgesic effect previously achieved by using the greater dosage, such as the greater daily dose, of said opioid). Opioid reduction may be assessed, for example, by using biomarkers for metabolites of opioids or by using patient's self-reported opioid intake.
The term “PEG scale”, as used herein, refers to the PEG three-item scale (i.e. as defined e.g. in http://mytopcare.org/wp-content/uploads/2013/06/PEG-Pain-Screening-Tooltpdf, Krebs E E, et. al. in J. Gen. Intern. Med, 2009, 24, 733-8, incorporated by reference herein). It is a 3 items clinical interview that: i) rates from 1 to 10 “Pain average” (P), ii) rates from 1 to 10 “interference with Enjoyment of life” (E), and iii) rates from 1 to 10 “interference with General activity. It has been shown to be sensitive to change, and can differentiate between subjects with and without pain improvement.
In particular, the term “for use in reversing opioid analgesic tolerance associated with chronic opioid use in chronic pain”, as used herein, is to be understood as “for use in a treatment to reverse opioid analgesic tolerance associated with chronic opioid use in chronic pain” or as “for use in a method of treatment to reverse opioid analgesic tolerance associated with chronic opioid use in chronic pain”.
The term “equianalgesic dose of another opioid”, as used herein, is to be understood as morphine milligram equivalent MME/day, as calculated, for example, according to the United States Department of Health and Human Services Centers for Disease Control and Prevention guideline (e.g., see, https://www.cdc.gov/drugoverdose/pdf/calculating_total_daily_dose-a.pdf; the entire contents of which are incorporated herein by reference), whereby morphine equivalent dosing is calculated on the basis of the following conversion factors:

| OPIOID (doses in mg/day | |
|---|---|
| except where noted) | CONVERSION FACTOR |
| Codeine | 0.15 |
| Fentanyl transdermal | 2.4 |
| (in mcg/hr) | |
| Hydrocodone | 1 |
| Hydromorphone | 4 |
| Methadone | |
| 1-20 mg/day | 4 |
| 21-40 mg/day | 8 |
| 41-60 mg/day | 10 |
| ≥61-80 mg/day | 12 |
| Oxycodone | 1.5 |
| Oxymorphone | 3 |
| Morphine | 1 |

Thus 50 MME/day refers to, for example, 50 mg of hydrocodone, 33 mg of oxycodone or 12 mg of methadone, per day; and 90 MME/day refers to, for example, 90 mg of hydrocodone, 60 mg of oxycodone or about 20 mg of methadone, per day.
The term MME refers to milligram equivalent dose of morphine.
The term “to reduce the risk of opioid-related overdose associated with chronic opioid use in chronic pain”, as used herein, is to be understood as a decrease in the risk of overdose associated with chronic opioid use for a patient with chronic pain. The risk for overdose may be assed, for example, according to the Pasero Opioid-induced Sedation Scale (POSS; e.g. in Pasero C, McCaffery M. Pain Assessment and Pharmacologic Management. St Louis: Mosby/Elsevier; 2011. Section IV, Opioid Analgesics; p.277-622), for example, compared to placebo.
The term “to reduce opioid consumption in chronic pain”, “reduces opioid consumption in chronic pain” or “reducing opioid consumption in chronic pain”, as used herein, as used herein, refers to reduction of opioid consumption in a patient with chronic pain. In one embodiment, it is to be understood as reduction of the opioid dosage (e.g. reduction of daily opioid dose) that a patient with chronic pain, who is a chronic opioid user, as defined herein, is being administered. In another embodiment, it is to be understood as a reduction in the disease status of opioid use (e.g. from moderate status to mild status, according to DMS-V diagnostic criteria for opioid use disorder). In particular, the reduction of opioid consumption in a patient with chronic pain is with maintained analgesic effect.
In particular, “reducing opioid consumption in chronic pain” or “reduction of opioid consumption in a patient with chronic pain” refers to a 50% opioid reduction of the original opioid dosage, such as a 50% opioid reduction of the original opioid dosage with a starting opioid dosage of form 60 mg to 100 mg oral morphine/day or daily equianalgesic dose of another opioid; for example a 50% opioid reduction of the original opioid dosage over a 5 weeks period, such as a 50% opioid reduction over a 5 weeks period of the original opioid dosage with a starting opioid dosage of form 60 mg to 100 mg oral morphine/day or daily equianalgesic dose of another opioid.
In particular, the term “opioid dosage”, as used herein, refers to daily opioid dose. The term “daily opioid dose”, as used herein, refers to mg oral morphine/day or daily equianalgesic dose of another opioid (i.e. morphine milligram equivalent MME/day). In one embodiment, the daily opioid dose is, for example. >50 mg oral morphine/day or daily equianalgesic dose of another opioid (i.e. morphine milligram equivalent MME/day), such as of from 60 mg oral morphine/day or daily equianalgesic dose of another opioid (e.g. hydrocodine or oxycodone) to 100 mg oral morphine/day or daily equianalgesic dose of another opioid (e.g. hydrocodine or oxycodone).
In particular, the daily opioid dose being administered to the chronic pain patient is decreased by 50% or more, such as a decrease by 70% or more, for example by 90% or more. In another embodiment, the daily opioid dose being administered to the chronic pain patient is decreased by 50% over a 5 weeks period, such as a 50% opioid reduction over a 5 weeks period of the original opioid dosage with a starting opioid dosage of form 60 mg to 100 mg oral morphine/day or daily equianalgesic dose of another opioid. Opioid reduction is assessed, for example, by using biomarkers for metabolites of opioids or by using patient's self-reported opioid intake (e.g. by using the Timeline Follow Back Opioid Self-reported Diary)].
In particular, the term “for use in reducing opioid consumption in chronic pain”, as used herein, is to be understood as “for use in a treatment to reduce opioid consumption in chronic pain” or as “for use in a method of treatment to reduce opioid consumption in chronic pain”.
In particular, the term “for use in the reduction of opioid consumption in chronic pain”, as used herein, is to be understood as “for use in a treatment for the reduction of opioid consumption in chronic pain” or as “for use in a method of treatment for the reduction of opioid consumption in chronic pain”.
The term “chronic pain”, as used herein, is to be understood as defined, for example, by the American Chronic Pain Association (ACPA) Resource Guide to Chronic Pain Management, 2017 Edition, namely as ongoing or recurrent pain, lasting beyond the usual course of acute illness or injury or more than 6 months, and which adversely affects the individual's well-being. Typically, chronic pain is classified by pathophysiology (the functional changes associated with or resulting from disease or injury), as nociceptive (due to ongoing tissue injury), neuropathic (resulting from damage to the brain, spinal cord or peripheral nerves) or a mixture of theses. The term “in chronic pain”, as used herein, may be understood as “by a patient with chronic pain” or “of a patient with chronic pain”.
The term “non-malignant chronic pain”, as used herein, refers to non-cancer chronic pain, which comprises, for example, post-herperic neuralgia, degenerative joint disorder (DJD; osteoarthritis), diabetic neuropathy, neuroma pain, central pain, sympathetic dystrophy, chronic musculoskeletal pain, migraine, fibromyalgia, rheumatoid arthritis, spinal stenosis and low back pain. In particular, non-malignant chronic pain refers to chronic post-surgical neuropathic pain. In another embodiment, non-malignant chronic pain refers to back pain, such as chronic low back pain. The term “low back pain” or “chronic low back pain”, as used herein, refers to pain that persists for 12 weeks or longer, even after an initial injury or underlying cause of acute low back pain has been treated (e.g. see, for example, North America Spine Society Diagnosis (NASS) and Treatment of Low-Back Pain Evidence-Based Guideline, 2016; or, Guideline for the Evaluation and Management of Low Back Pain: Evidence Review, R. Chou, L. Hoyt Huffman, American Pain Society).
The term “malignant chronic pain”, as used herein, refers to cancer chronic pain (e.g. chronic pain syndromes as described in Appendix E of the Interagency Guideline on Prescribing Opioids for Pain, 3^{rd} Edition, June 2015, the entire contents of which are incorporated herein by reference).
As used herein, the term “treat”, “treating” or “treatment” in connection to a disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In one embodiment, the term “method for the treatment of”, as used herein, refers to “method to treat”.
The term “elderly patient”, as used herein, refers to a patient sixty-five years of age or older.
As used herein, “bid”, “b.i.d” or “b.i.d.” refers to taken twice (two times) a day (e.g. 200 mg b.i.d=400 mg/day), for example taken in the morning and evening (e.g. separated by approximately 12 hour intervals).
The term “patient”, as used herein, refers to a subject who is diseased and would benefit from the treatment. The term “chronic pain patient” or “patient with chronic pain”, as used herein, refers to a subject with chronic pain (i.e. diagnosed with chronic pain, as defined herein). In one embodiment, the term “patient”, refers to a subject diagnosed with chronic pain, as defined herein, who is a chronic opioid user and is opioid dependent (i.e. who has withdrawal symptoms upon opioid cessation, but who does not meet DSM-V opioid use disorder criteria). In another embodiment, the term “patient”, refers to a subject diagnosed with chronic pain, as defined herein, who meets mild or moderate DSM-V opioid use disorder criteria.
As used herein, the term “subject” refers to a mammalian organism, preferably a human being (male or female).
As used herein, a subject is “in need of” a treatment if such subject (patient) would benefit biologically, medically or in quality of life from such treatment.
The term “a therapeutically effective amount” of a compound of the present invention refers to an amount of a compound of the present invention that will elicit the biological or medical response of a subject, for example, ameliorate symptoms, alleviate conditions, etc.
The term “opioid use disorder” or “OUD” as used herein refers to DSM-5 criteria for opioid use disorder (i.e. according to the Diagnostic and Statistical Manual of Mental Disorders. 5th Edition, Washington, D.C.: American Psychiatric Association, 2013; the entire contents of which are incorporated herein by reference), which may be separated into the following three categories: mild (i.e. presence of 2 to 3 symptoms, defined with reference to DSM-5 criteria), moderate (i.e. presence of 4 to 5 symptoms, defined with reference to DSM-5 criteria) and severe (i.e. presence of 6 or more symptoms, defined with reference to DSM-5 criteria). Thus, as used herein, the term “opioid use disorder” is defined as a problematic pattern of opioid use leading to clinically significant impairment or distress, as manifested by at least two of the following, occurring within a 12-month period:

1) Opioids are often taken in larger amounts or over a longer period than was intended.

2) There is a persistent desire or unsuccessful efforts to cut down or control opioid use.

3) A great deal of time is spent in activities necessary to obtain the opioid, use the opioid, or recover from its effects.

4) Craving, or a strong desire or urge to use opioids.

5) Recurrent opioid use resulting in a failure to fulfill major role obligations at work, school, or home.

6) Continued opioid use despite having persistent or recurrent social or interpersonal problems caused or exacerbated by the effects of opioids.

7) Important social, occupational, or recreational activities are given up or reduced because of opioid use.

8) Recurrent opioid use in situations in which it is physically hazardous.

9) Continued opioid use despite knowledge of having a persistent or recurrent physical or psychological problem that is likely to have been caused or exacerbated by opioids.

10) Tolerance, as defined by either of the following: a) A need for markedly increased amounts of opioids to achieve intoxication or desired effect; b) A markedly diminished effect with continued use of the same amount of opioid.

11) Withdrawal, as manifested by either of the following:
a) The characteristic opioid withdrawal syndrome (refer to DSM-5 criteria A and B of the criteria set for opioid withdrawal).
b) Opioids (or a closely related substance) are taken to relieve or avoid withdrawal symptoms.
The term “pharmaceutical composition” is defined herein to refer to a mixture or solution containing at least one active ingredient or therapeutic agent to be administered to a subject, in order to treat a particular condition (i.e. disease, disorder or condition or at least one of the clinical symptoms thereof) affecting the subject.

The Opioid Craving Scale (OCS) is a modification of the Cocaine Craving Scale and is a short 3-item reported outcome that is used to assess craving experience during opioid consumption. The 3-item scale comprises a visual analogue measure from 1 to 10 and is used to address how much an opioid user craves an opiate; how strong a desire an opioid user desired to use an opiate on a previous day; and if the opioid user has a desire to take opioids at the same time on the current day. Based on the responses on the 1 to 10 scale for the three items, each one-unit increase in the score was associated with a 17% greater odds of using opioids in the following week (e.g. McHugh R K et al., in Assessing craving and its relationship to subsequent prescription opioid use among treatment-seeking prescription opioid dependent patients, Drug and Alcohol Dependency, 2014, Dec. 1; 145: 121-6).
The Clinical Opiate Withdrawal Scale (COWS) consists of an 11-item scale that is administered by a physician and is used to rate the more common signs and symptoms of opiate withdrawal and review these symptoms over a period of time (e.g. Wesson D R, et al., in Journal of Psychoactive Drugs, 2003, Apr-Jun; 35(2): 253-9). The added score for the entire 11-item scale can assist physicians to assess the stage or severity for opiate withdrawal that a user is experiencing and also provide an indication of the degree of physical dependence on an opiate.
The Numerical Opioid Side Effect (NOSE) assessment (e.g. in Smith H. S., et al., Med Clin North Am., 2007, 91(2):213-228) is a 10-item scale of common opiate-specific side effects. Each side effect is represented by an analogue scale from 0 to 10, with zero indicating a side effect is not present and, 10 being a side effect that is as bad as the opiate user can image.
The Numeric Rating Scale (NRS; e.g. in Childs J. D., et al., Spine, 2005; 30:1331-4)) assesses pain intensity on a scale of from 0 (no pain) to 10 (worst imaginable pain).
The Columbia Suicide Severity Rating Scale (C-SSRS; e.g. in Posner K et al., Am. J. Psychiatry; 2011, 168:1266-1277) is a suicidal ideation and behavior rating scale to evaluate suicide risk.
As used herein, the term “pharmaceutically acceptable excipient” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 22^{nd} Ed. Mack Printing Company, 2013, pp. 1049-1070). Except insofar as any conventional carrier is incompatible with the active ingredient, its use in the therapeutic or pharmaceutical compositions is contemplated.
The terms “drug”, “active substance”, “active ingredient”, “pharmaceutically active ingredient”, “active agent” or “therapeutic agent” are to be understood as meaning a compound in free form or in the form of a pharmaceutically acceptable salt, in particular compounds of the type specified herein.
The term “combination” or “pharmaceutical combination” refers to either a fixed combination in one unit dosage form (e.g., capsule or tablet), non-fixed combination, or a kit of parts for the combined administration where a compound of the present invention and one or more combination partner (e.g. another drug as specified herein, also referred to as further “pharmaceutical active ingredient”, “therapeutic agent” or “co-agent”) may be administered independently at the same time or separately within time intervals, especially where these time intervals allow that the combination partners show a cooperative, e.g. synergistic effect. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. the compound of the present invention and one or more combination partners, are both administered to a patient simultaneously in the form of a single entity or dosage. The term “non-fixed combination” means that the active ingredients, e.g. a compound of the present invention and one or more combination partners, are both administered to a patient as separate entities either simultaneously or sequentially with no specific time limits, wherein such administration provides therapeutically effective levels of the two compounds in the body of the patient.
The compound of the present invention may be administered separately, by the same or different route of administration, or together in the same pharmaceutical composition as the other agents. In the combination therapies of the invention, the compound of the invention and the other therapeutic agent may be manufactured and/or formulated by the same or different manufacturers. Moreover, the compound of the invention and the other therapeutic may be brought together into a combination therapy: (i) prior to release of the combination product to physicians (e.g. in the case of a kit comprising the compound of the invention and the other therapeutic agent); (ii) by the physician themselves (or under the guidance of the physician) shortly before administration; (iii) in the patient themselves, e.g. during sequential administration of the compound of the invention and the other therapeutic agent.
In particular, reference to a combination with a further active agent, as used herein (e.g. in any of embodiments herein above, or in any of the claims, herein below), refers, for example, to a combination with at least one further active agent, for example, selected from the group consisting of an antidepressant (e.g. a tricyclic antidepressant, such as amitriptyline, nortriptyline, doxepin, desipramine, imipramine, protriptyline, trimipramine, clomipramine), a serotonin norepinephrine reuptake inhibitor (e.g. duloxetine, venlafaxine, desvenlafaxine, milnacipran, levomilnacipran), a serotonine reuptake inhibitor (e.g. fluoxetine, setraline, paroxetine, fluvoxamine, citalopram, escitalopram, vilazodone, vortioxetine), an anticonvulsant (e.g. gabapentin, pregabalin, carbamazepine, valproic acid, phenytoin, lamotrigine, tiagabine, lacosamide, topiramate, levetiracetam, oxacarbazepine, zonisamide), a non-steroidal anti-inflammatory drug (NSAID; such as naproxen, ibuprofen, meloxicam, diclofenac, fenoprofen, flurbiprofen, diflunisal, etodolac, nabumetone, ketoprofen, piroxicam, sulindac, tolmetin, ketorolac, mefenamic, oxaprozin), a proton pump inhibitor (e.g. omeprazole, pantoprazole, lansoprazole, dexlansoprazole, esomeprazole, rabeprazole), a H2 receptor antagonist (e.g. famotidine, nizatidine, ranitidine, cimetidine), an NMDA inhibitor (e.g. ketamine, amantadine, mematine), NO-NSAID, a COX-2 selective inhibitor, a cannabinoid agonist, a nitric oxide donor, a beta adrenergic agonist, an alpha-2 agonist, a selective prostanoid receptor antagonist, a local anesthetic (e.g. capsaicin, lidocaine), a purinergic P2 receptor antagonist, a neuronal nicotinic receptor agonist, a calcium channel antagonist, a sodium channel blocker (e.g. mexiletine, flecainide), a superoxide dismutase mimetic, a p38 MAP kinase inhibitor, a TRPVI agonist, a glycine receptor antagonist, a corticosteroid, and acetaminophen; or pharmaceutically acceptable salts thereof.
The term “immediate release form” refers to a pharmaceutical composition designed to release the active substance immediately upon in vivo administration.
The term “modified release form” refers to a pharmaceutical composition which releases the active substance not immediately, but offers a sustained, retard, continuous, gradual, prolonged or pulsatile release and therefore alters drug plasma levels distinctively versus an immediate release form. The term “modified release form” encompasses forms that are described as controlled-release form, sustained-release form, extended-release form, and long-acting form; in particular a sustained-release form.
As used herein, the term “a,” “an,” “the” and similar terms used in the context of the present invention (especially in the context of the claims) are to be construed to cover both the singular and plural unless otherwise indicated herein or clearly contradicted by the context.
The use of any and all examples, or exemplary language (e.g. “such as”) provided herein is intended merely to better illuminate the invention and does not pose a limitation on the scope of the invention otherwise claimed.
As used herein, the compound of the invention, alternatively named Compound (I), as used herein above and below, is the mGluR5 antagonist (−)-(3aR,4S,7aR)-4-Hydroxy-4-m-tolylethynyl-octahydro-indole-1-carboxylic acid methyl ester, also named (−)-(3aR,4S,7aR)-4-Hydroxy-4-[2-(3-methylphenyl)ethynyl]perhydroindole-1-carboxylic acid methyl ester, also known as mavoglurant, of formula:

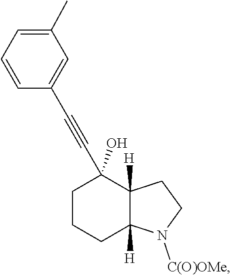

which can be e.g. prepared as described in WO2003/047581, e.g., in Example 1, or as described in WO2010/018154. WO2003/047581, which is incorporated herein by reference, also describes its in-vitro biological data, as per page 7. As used herein, “mavoglurant” refers to the free form. In particular, mavoglurant is in the free form. As used herein, the term “mavoglurant, or a salt thereof, such as a pharmaceutically acceptable salt thereof”, as used in the context of the present invention (especially in the context of the any of the embodiments, above or below, and the claims) is thus to be construed to cover both the free form and a pharmaceutically acceptable salt thereof, unless otherwise indicated herein.
In one embodiment, Compound (I) is also intended to represent isotopically labeled forms. Isotopically labeled compounds have structures depicted by the formula above except that one or more atoms are replaced by an atom having a selected atomic mass or mass number. Isotopes that can be incorporated into the compound of the invention include, for example, isotopes of hydrogen, namely the compound of formula:

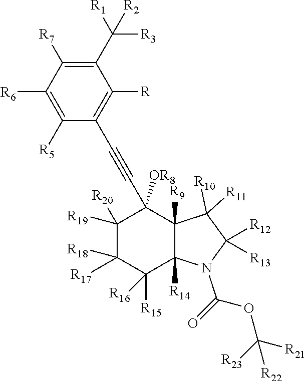

wherein each R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂ and R₂₃ is independently selected from H or deuterium; provided that there is at least one deuterium present in the compound. In other embodiments there are multiple deuterium atoms present in the compound.

As used herein, the compound of the invention, alternatively named Compound (II), as used herein above and below, is the mGluR5 antagonist known as 9-cyclopropyl-10-fluoro-2-(4-(methoxymethyl)-1H-imidazol-1-yl)-7,8-dihydro-[1,4]diazepino[7,1-a]isoquinolin-5(4H)-one, of formula:

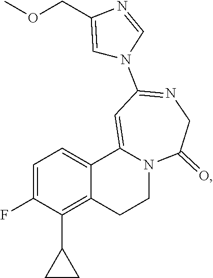 [SMILES: COCc1cn(C2=NCC(=O)N3CCc4c(ccc(F)c4C4CC4)C3=C2)cn1]

which can be e.g. prepared as described in WO2014/030128, e.g., in Example 96-2, which is incorporated herein by reference, and which also describes its in-vitro biological data, as per page 203. As used in the context of the present invention (especially in the context of the any of the embodiments, above or below, and the claims), the term “Compound (II)” is to be construed to cover both the free form and a pharmaceutically acceptable salt thereof, unless otherwise indicated herein.

In one embodiment, Compound (II) is also intended to represent isotopically labeled forms. Isotopically labeled compounds have structures depicted by the formula above except that one or more atoms are replaced by an atom having a selected atomic mass or mass number. Isotopes that can be incorporated into the compound of the invention include, for example, isotopes of hydrogen, namely the compound of formula:

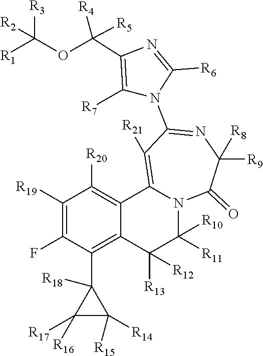

wherein each R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀ and R₂₁ is independently selected from H or deuterium; provided that there is at least one deuterium present in the compound. In other embodiments there are multiple deuterium atoms present in the compound. Isotopically labeled forms of Compound (II) are for example:

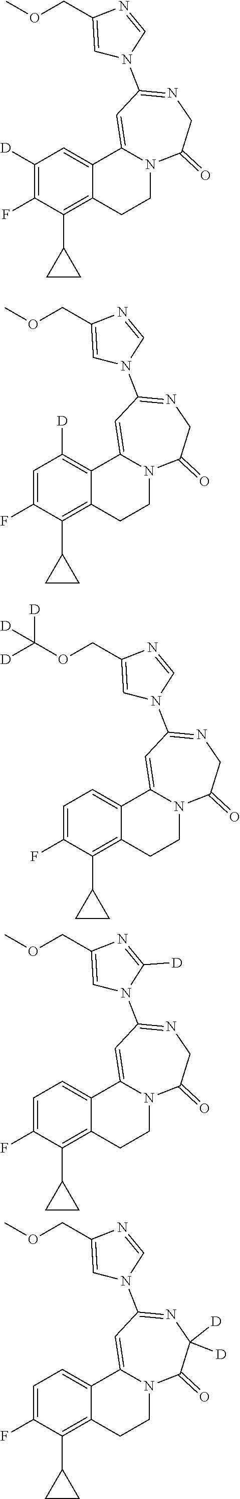

As used herein, the compound of the invention, alternatively named Compound (III), as used herein above and below, is the mGluR5 antagonist known as 2-methyl-6-(phenylethynyl)pyridine, also known as MPEP, of formula:

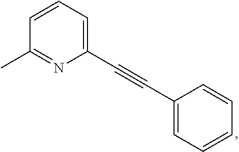

whose CAS Number is 96206-92-7 and it is commercially available. As used in the context of the present invention (especially in the context of the any of the embodiments, above or below, and the claims), the term “Compound (III)” is to be construed to cover both the free form and a pharmaceutically acceptable salt thereof, unless otherwise indicated herein.

In one embodiment, Compound (III) is also intended to represent isotopically labeled forms. Isotopically labeled compounds have structures depicted by the formula above except that one or more atoms are replaced by an atom having a selected atomic mass or mass number. Isotopes that can be incorporated into the compound of the invention include, for example, isotopes of hydrogen, namely the compound of formula:

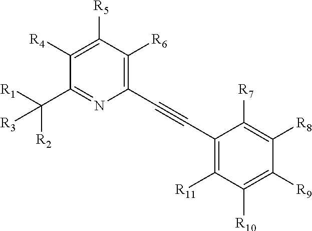

wherein each R₁, R₂, R₃, R₄, R₅, R₆, R₇, R₈, R₉, R₁₀ and R₁₁ is independently selected from H or deuterium; provided that there is at least one deuterium present in the compound. In other embodiments there are multiple deuterium atoms present in the compound.
Incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of the compound of the invention. The concentration of deuterium, may be defined by the isotopic enrichment factor. The term “isotopic enrichment factor” as used herein means the ratio between the isotopic abundance and the natural abundance of a specified isotope. If a substituent in the compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in analogous manner as described for deuterium.
Other examples of isotopes that can be incorporated into the compound of the invention include isotopes of hydrogen, other than deuterium, carbon, nitrogen, oxygen, and fluorine such as ³H, ¹¹C, ¹³C, ¹⁴C, ¹⁵N, ¹⁸F respectively. Accordingly, it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. The isotopically-labeled compounds can generally be prepared by conventional techniques known to those skilled in the art or by processes analogous to those described preparation of the compound of the invention by using an appropriate isotopically-labeled reagent& in place of the non-labeled reagent previously employed.
As used herein, the terms “free form” or “free forms” refers to the compound in non-salt form, such as the base free form or the acid free form of a respective compound, e.g. the compounds specified herein [e.g. Compound (I), Compound (II), Compound (III) or further pharmaceutical active ingredient, for example, as defined herein].
As used herein, the terms “salt”, “salts” or “salt form” refers to an acid addition or base addition salt of a respective compound, e.g. the compounds specified herein [e.g. Compound (I), Compound (II), Compound (III) or further pharmaceutical active ingredient, for example, as defined herein]. “Salts” include in particular “pharmaceutically acceptable salts”. The term “pharmaceutically acceptable salts” refers to salts that retain the biological effectiveness and properties of the compounds and, which typically are not biologically or otherwise undesirable.
Pharmaceutically acceptable acid addition salts can be formed with inorganic acids and organic acids.
Inorganic acids from which salts can be derived include, for example, hydrochloric acid, hydrobromic acid, sulfuric acid, nitric acid, phosphoric acid, and the like.
Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.
Pharmaceutically acceptable base addition salts can be formed with inorganic and organic bases.
Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.
Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.
Pharmaceutically acceptable salts can be synthesized from a basic or acidic moiety, by conventional chemical methods. Generally, such salts can be prepared by reacting the free acid forms of the compound with a stoichiometric amount of the appropriate base (such as Na, Ca, Mg, or K hydroxide, carbonate, bicarbonate or the like), or by reacting the free base form of the compound with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 22^{nd} edition, Mack Publishing Company (2013); and in “Handbook of Pharmaceutical Salts: Properties, Selection, and Use” by Stahl and Wermuth (Wiley-VCH, Weinheim, 2011, 2^{nd} edition).
The compounds specified herein [e.g. Compound (I), Compound (II), Compound (III) or the further pharmaceutical active ingredient, for example, as defined herein] can be administered by conventional route, in particular orally, such as in the form of tablets or capsules, which can be manufactured according to pharmaceutical techniques as known in the art (for example in “Remington Essentials of Pharmaceutics, 2013, 1^{st} Edition, edited by Linda Felton, published by Pharmaceutical Press 2012, ISBN 978 0 85711 105 0; in particular Chapter 30), wherein pharmaceutical excipients are, for example, as described in “Handbook of Pharmaceutical Excipients, 2012, 7^{th} Edition, edited by Raymond C. Rowe, Paul J. Sheskey, Walter G. Cook and Marian E. Fenton, ISBN 978 0 85711 027 5″. In particular, WO2014/199316 describes formulations comprising mavoglurant, in particular modified release formulations thereof, and is incorporated herein by reference, more particularly the Examples, the preferred embodiments and claims therein.
The pharmaceutical composition, or combination of the present invention, can be in a unit dosage form (e.g. tablet or capsule) comprising an amount ranging of from 1 mg to 300 mg, in particular of from 1 mg to 200 mg, such as of from 1 mg to 100 mg, of the compound specified herein (e.g. mavoglurant), referring to an amount of the free form of the compound, and, if a salt thereof is used, the amount will be adapted accordingly. In particular, mavoglurant is in the free form.

For the above-mentioned uses/treatment methods the appropriate dosage may vary depending upon a variety of factors, such as, for example, the age, weight, sex, the route of administration or salt employed. In patients with, for example, of from 50-70 kg body weight, an indicated daily dosage is, for example, 200 mg/b.i.d of mavoglurant, or of from 1 mg/day to 100 mg/day of Compound (II), referring to amounts of the free form, and if a salt thereof is used the amount will be adapted accordingly.

### ABBREVIATIONS

° C.=degree Celsius
bid=b.i.d=b.i.d.=twice (two times) a day
cm=centimeter
DSM 5=Diagnostic and Statistical Manual of Mental Disorders, 5th Ed
i.p.=intraperitoneal
Kg=kilogram
mg=milligram
p.o.=by mouth, orally
PEG=assessment of pain intensity (P), enjoyment of life (E), general activity (G)
NRS=Numeric Rating Scale
TLFB-Opioid=Timeline Follow Back Opioid Self-Reported Diary
COWS=Clinical Opiate Withdrawal Scale
OCS=Opioid Craving Scale
NOSE=Numerical Opioid Side Effect
DSM-5=DMS-V=Diagnostic and Statistical Manual of Mental Disorders 5th Edition
QTcF=QT interval corrected by Fridericia's formula
QTcB=QT interval corrected by Bazett's formula
ECG=Electrocardiogram
C-SSRS=Columbia Suicide Severity Rating Scale
AST=aspartate aminotransferase
ALT=alanine aminotransferase
GGT=Gamma-glutamyl transferase
ULN=upper limit of normal
OUD=Opioid Use Disorder
hCG=Human chorionic gonadotropin
FDA=Food and Drug Administration
QT=time between the start of the Q wave and the end of the T wave
T wave=positive deflection after each QRS complex
ST wave=time between the start of the S wave and the end of the T wave

### EXAMPLES

The following Examples serve to illustrate the invention without limiting the scope thereof. The term “Compound (I)” (i.e. mavoglurant), as used in the context of these examples, refers to the free form. The term “Compound (II)” {i.e. 9-cyclopropyl-10-fluoro-2-(4-(methoxymethyl)-1H-imidazol-1-yl)-7,8-dihydro-[1,4]diazepino[7,1-a]isoquinolin-5(4H)-one}, as used in the context of these examples, refers to the free form. The term “Compound (III)” (i.e. MPEP), as used in the context of these examples, refers to the free form.

### Example 1: Opiate Tolerance Test (Hot Plate) in the Mouse

The method, which detects tolerance to analgesic activity, follows that described by Fernandes et al (Naunyn-Schmiedeberg's Arch. Pharmacol., 297, 53-60, 1977).

Mice were placed onto a hot metal plate maintained at 54° C. surrounded by a Plexiglas cylinder (height: 13 cm; diameter: 19 cm) (Bioseb: model PE34). The latency to the first foot-lick was measured (maximum: 30 seconds). Morphine (32 mg/kg i.p.) or vehicle were administered twice daily for 6 days (at about 9:00 and 15:00), and then administered with morphine 8 mg/kg i.p. (or saline) 30 minutes before the test on Day 7 to assess morphine tolerance.

The test substances were each evaluated at 3 doses (1, 3 and 10 mg/kg for Compound (I) and 0.3, 1 and 3 mg/kg for Compound (II), administered in morphine tolerant mice p.o. acutely 60 minutes before the test on Day 7. The experiment included appropriate control groups to assess the potential effects of the test substances on morphine tolerance. Compound (III) was evaluated at 1 dose (10 mg/kg), administered under the same experimental conditions and was used as comparison substance.

10 mice were studied per group. The test was performed blind. Data were analysed by comparing treated groups with appropriate controls using unpaired Student's t tests.

Results:

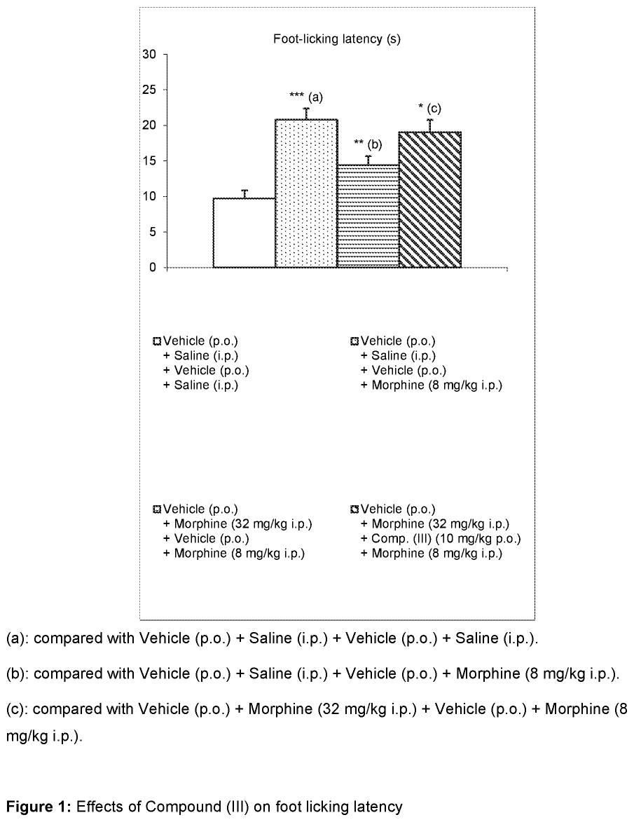

Compound (III) (10 mg/kg), administered p.o. 60 minutes before the test on Day 7 in animals receiving vehicle p.o. and morphine (32 mg/kg) i.p. twice daily from Day 1 to Day 6, significantly increased the foot-licking latency, as compared with animals receiving morphine from Day 1 to Day 6 (+32%, p<0.05) (FIG. 1).

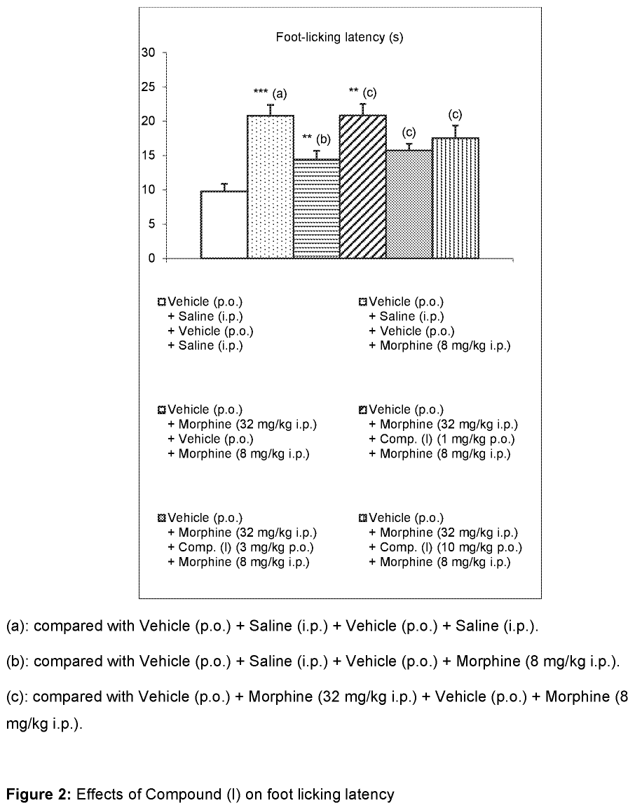

Compound (I) (1 mg/kg) administered p.o. 60 minutes before the test on Day 7 in animals receiving vehicle p.o. and morphine (32 mg/kg) i.p. twice daily from Day 1 to Day 6, significantly increased the foot-licking latency, as compared with animals receiving morphine from Day 1 to Day 6 (+44%, p<0.01). It had no significant effects at 3 or 10 mg/kg (FIG. 2).

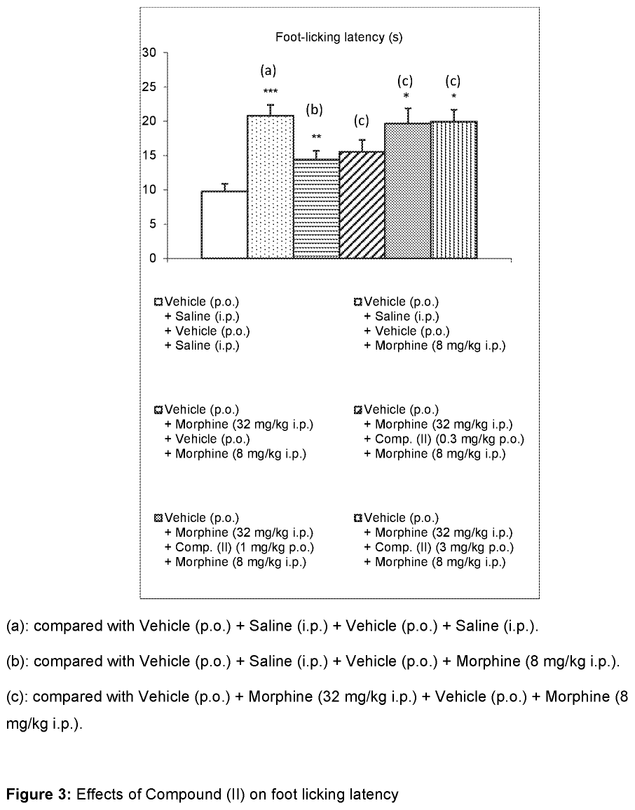

Compound (II) (3 mg/kg) administered p.o. 60 minutes before the test on Day 7 in animals receiving vehicle p.o. and morphine (32 mg/kg) i.p. twice daily from Day 1 to Day 6, significantly increased the foot-licking latency, as compared with animals receiving morphine from Day 1 to Day 6 (+38%, p<0.05). It had the same tendency at 1 mg/kg (+37%, p=0.0555) but had no effects at 0.3 mg/kg (FIG. 3).

Conclusion:

These results suggest that Compound (I) (1 mg/kg), Compound (II) (3 mg/kg) and Compound (III) (10 mg/kg), administered p.o. acutely significantly decrease the expression of morphine tolerance.

### Example 2: Clinical Testing [Compound (I)]

| Objective(s) | Endpoint(s) |
|---|---|
| Primary objective(s) | Endpoint(s) for primary objective(s) |
| To evaluate the efficacy of | Proportion of patients with a 50% |
| Compound (I) in chronic | reduction in consumption using the |
| back-and post-surgical | Timeline Follow Back-Opioid |
| pain patients in | self-reported diary (TLFB). |
| achieving a clinical | |
| reduction in opioid intake. | |
| Secondary objective(s) | Endpoint(s) for secondary objective(s) |
| Suicidality. | Columbia-Suicide Severity Rating |
| | Scale (C-SSRS). |
| Change in status for pain. | Numeric Rating Scale (NRS). |
| Change in the disease status | DSM-5 diagnostic criteria for |
| of opioid use disorder. | opioid use disorder. |
| Adverse outcomes of opioid | Numerical Opioid Side Effect |
| use disorder. | (NOSE) Assessment. |
| Cravings associated with | Opioid Craving Scale (OCS). |
| opioid use. | |
| Withdrawal symptoms associated | Clinical Opiate Withdrawal |
| with opioid use disorder. | Scale (COWS). |
| To evaluate the efficacy of | Proportion of patients with a |
| Compound (I) in chronic back- | 20% reduction in consumption |
| and post-surgical pain patients | or a 70% reduction in |
| in achieving reduction in | consumption using the Timeline |
| opioid intake. | Follow Back - Opioid self- |
| | reported diary. |
| Change in Pain, Activity and | Pain, Activity and Enjoyment |
| Enjoyment. | (PEG) Scale |

Study Design

This study is a randomized, subject- and investigator-blinded, placebo-controlled, parallel group study to evaluate the efficacy of Compound (I) in 80 patients with chronic low back- and post-surgical pain patients in reducing opioid intake (hydrocodone, oxycodone) in subjects who have used opioids for less than two years. The study involves out-patient visits to the subjects' usual pain clinic and consists of 3 epochs: Screening with Baseline; Treatment; followed by the Treatment Follow-Up. The total duration for a subject may be up to 122 days (approximately 4 months including screening and baseline) depending on the elected percent opioid reduction.

Screening/Baseline Epoch

The screening/baseline epoch last for 14 days and consists of:
1. Obtaining informed consent
2. Initial diagnostic criteria of opioid tolerance using the DSM-V criteria.
3. Hematology/biochemistry laboratory safety assessment including pregnancy test for pre-menopausal female subjects
4. Medical history, physical examination and vitals, current medications and risk for suicidality

Treatment Epoch

Subjects fulfilling all eligibility criteria will be randomly allocated to either Compound (I) arm or placebo matched arm in a ratio of 1:1. Study drug treatment should start on Day 1 with total treatment lasting approximately 93 days during which time subjects will reduce their opioid dose by a minimum of 10% of their starting dose per week, in consultation with their study physician. Subjects will titrate through 50 mg, 100 mg and 200 mg bid every 4 days (see table 1) to reach 400 mg total daily dose by Day 12. During this titration phase, subjects will be monitored for safety as the study drug dose is increased, which will include visits on Day 1, 5 and 9, to ensure that subjects have reached the maintenance dose of 400 mg per day without tolerability issues.

PK samples will be collected at predose and 4±1 hours post dose on day 13, 55 and 69, respectively and a predose PK sample on day 83. On the day of PK sampling the subjects will take the Compound (I) dose at the site as a predose sampling needs to take place before the dosing.

TABLE 1
Compound (I) Titration Schedule

| Day of Study | Treatment Dose | Total Daily Treatment Dose |
|---|---|---|
| 1-4 | 50 mg bid | 100 mg Compound (I) or |
| | Compound (I) or Placebo | Placebo per day |
| 5-8 | 100 mg bid | 200 mg Compound (I) or |
| | Compound (I) or Placebo | Placebo per day |
| 9-12 | 200 mg bid | 400 mg Compound (I) or |
| | Compound (I) or Placebo | Placebo per day |

Following titration up to 400 mg per day, subjects will continue with a daily dose of 400 mg of Compound (I) (200 mg b.i.d) or placebo from Day 13 through Day 82. During this time, subjects will reduce their opioid consumption by a minimum of 10% per week in consultation with their study physician until they reach a daily opioid consumption of at least 50% of their starting opioid MME dose. Subjects who are unable to reduce may remain at the current dose of opioid that they achieved during the preceeding week of the study. Subjects can, under the direction of their study physician, reduce their opioid consumption to more than 50%. Subjects may also aim to reduce their opioid intake by utilizing alternative methods of therapy. However, marijuana use for pain is not permitted under this protocol.

The starting Day for Compound (I) tapering is Day 83 with completion of tapering for Compound (I) at Day 90. Opioid reduction may extend through out the maintenance period of Compound (I) dosing assuming a conservative reduction of opioid of 10% per week or subjects can remain on the dose of opioid that they have achieved during their opioid reduction throughout the remaining Compound (I) maintenance period and tapering. The protocol is flexible to allow the physician and patients to elect to reduce opioid consumption that best suits the subject treatment and medical history. However, Compound (I) treatment is not flexible and should follow the assessment schedule for up-titration, maintenance and tapering. Subjects will taper their study drug (Compound (I) or Placebo) until they reach their last dose of Compound (I) or placebo (Table 2). Subjects will continue with the dose of opioid that they have achieved during the reduction process.

TABLE 2
Compound (I) tapering schedule

| Day of Study | Treatment Dose | Total Daily Treamtent Dose |
|---|---|---|
| 83-86 | 100 mg b.i.d | 200 mg per day |
| | Compound (I) or Placebo | Compound (I) or Placebo |
| 87-90 | 50 mg b.i.d | 100 mg per day |
| | Compound (I) or Placebo | Compound (I) or Placebo |
| 97 | End of Study follow up | |

Treatment Follow-Up

Subjects will return to the clinic on Day 97 for an end of treatment completion follow-up visit. Subjects must have tapered the study drug treatment prior to the end of treatment completion follow-up visit.

Dosing in Combination with Forced Opioid Reduction During the Study

Since patients may take different opioids (hydrocodone and oxycodone) in different dosages, these medications should be converted into Morphine Equivalent Dose (MED) and opioid reduction should be done using MED. As opioid reduction plans should be individualized to minimize symptoms of opioid withdrawal while maximizing pain treatment, it will be up to the investigator to reduce according to the criteria noted in the Oxford University Hospitals Guidance for Opioid Reduction in Primary Care (v1.1 Dec. 2017) for reference see Table 3.

TABLE 3
Example reduction schedule assuming a minimum 10% opioid
reduction per week with a starting dose of 100 MME

| | Daily Total Compound (I) | Daily Morphine Equivalent |
|---|---|---|
| Days of Study | Dose | Dose (10% Reduction) |
| 1-4 | 100 mg per day | 100 |
| 5-8 | 200 mg per day | 100 |
| 9-12 | 400 mg per day | 100 |
| 13-19 V | 400 mg per day | 90-80 |
| 20-26 | 400 mg per day | 80-70 |
| 27-33 V | 400 mg per day | 70-60 |
| 34-40 | 400 mg per day | 60-50 |
| 41-47 V | 400 mg per day | 50-45 |
| 48-54 | 400 mg per day | 45-40 |
| 55-61 V | 400 mg per day | 40-35 |
| 62-68 | 400 mg per day | 35-30 |
| 69-75 V | 400 mg per day | 30-25 |
| 76-82 | 400 mg per day | Last opioid dose achieved |
| 83-86 V | 200 mg per day | Last opioid dose achieved |
| 87-90 | 100 mg per day | Last opioid dose achieved |
| 97 | End of Study Follow-Up | |

Subjects are not mandated to reduce their opioid intake to zero MME. The study physician and the subject should determine the stopping point for opioid reduction.

Population

A total of approximately 80 patients, aged between 18 and 65 years of age (inclusive) with a diagnosis of Opioid Use Disorder will be enrolled in the study.

The investigator must ensure that all subjects being considered for the study meet the following eligibility criteria. No additional criteria should be applied by the investigator, in order that the study population will be representative of all eligible subjects.

Subject selection is to be established by checking through all eligibility criteria at screening and again at baseline to ensure that the subject remains eligible for entry into the study. A relevant record (e.g., checklist) of the eligibility criteria must be stored with the source documentation at the study site.

Deviation from any entry criterion excludes a subject from enrollment into the study.

Inclusion Criteria

Subjects eligible for inclusion in this study must meet all of the following criteria:
1. Healthy male and female subjects 18 to 65 years of age (inclusive) who can provide written informed consent.
2. Able to communicate well with the investigator, to understand and comply with the requirements of the study.
3. Prospective subjects should be taking opioids on a fixed dosing schedule (e.g., bid).
4. Documented evidence of increasing opioid doses without pain reduction or increase in pain intensity when taking the same amount of a drug in at least 4 weeks prior to screening.
5. Subjects must be in good health as determined by medical history and physical examination at screening.
6. Subjects with chronic low back pain or chronic post-surgical neuropathic pain present for more than 6 months after a traumatic or surgical event taking between 60 and 100 milligram morphine equivalent dose such as, for example, motor vehicle accident, fall, sports injury, knee or hip replacement. Subjects with mild to moderate osteoarthritis may be enrolled.
7. At screening, vital signs (systolic and diastolic blood pressure, pulse rate and respiratory rate) will be assessed in the sitting position and again (when required) in the standing position. Vital signs should be within normal limits determined by the Principal Investigator.
8. Patients with mild to moderate DSM-5 Criteria for Diagnosis of Opioid Use Disorder.

Exclusion Criteria

Subjects meeting any of the following criteria are not eligible for inclusion in this study.
1. Subject cannot anticipate any significant problems with transportation arrangements or available time to travel to the study site or have any plans to move within the next months to a location that would make continued participation in the study impractical.
2. Subjects should not be involved in any unresolved legal problems that could jeopardize continuation or completion in the study.
3. History of immunodeficiency diseases by medical record
4. History of chronic infection with Hepatitis B (HBV) or Hepatitis C (HCV) by medical history.
5. History or current diagnosis of ECG abnormalities indicating significant risk of safety for subjects participating in the study such as:
   Concomitant clinically significant cardiac arrhythmias, e.g. sustained ventricular tachycardia, and clinically significant second or third degree AV block without a pacemaker
   History of familial long QT syndrome or known family history of Torsades de Pointes
   Resting heart rate (physical exam or 12 lead ECG)<60 bpm
   Resting QTcF 450 msec (male) or 460 msec (female) at pre-treatment [screening]
      Note that if QTcB (Bazett) is the only available correction produced by the ECG machine, the investigator must calculate QTcF at screening and/or baseline (as applicable) per instructions in the Site Operations Manual.
      Note that sinus tachycardia, left axis deviation, and non-specific ST or T wave changes are not exclusionary.
   Use of agents known to prolong the QT interval unless they can be permanently discontinued for the duration of study
6. Score “yes” on item 4 or item 5 of the Suicidal Ideation section of the C-SSRS, if this ideation occurred in the past 6 months, or “yes” on any item of the Suicidal Behavior section, except for the “Non-Suicidal Self-Injurious Behavior” (item also included in the Suicidal Behavior section), if this behavior occurred in the past 2 years.
7. Has current diagnosis of Substance Use Disorder (according to the DSM-5) on alcohol or other stimulants, except physician-prescribed opioids.
8. Any surgical or medical condition which might significantly alter the absorption, distribution, metabolism, or excretion of drugs, or which may jeopardize the subject in case of participation in the study. The Investigator should make this determination in consideration of the subject's medical history and/or clinical or laboratory evidence of any of the following: Clinical laboratory values (including AST, ALT, total bilirubin or creatinine) considered as not clinically acceptable for OUD population, in the opinion of the Principal Investigator, at screening.
   Serum bilirubin must not exceed 1.5×ULN
   ALT, AST, GGT, must not exceed 3×ULN.
   Note: in the instance where a safety laboratory assessment at screening is outside of the range specified above, the assessment may be repeated once prior to randomization. If the repeat value remains outside of the specified ranges, the subject is excluded from the study.
9. History of hypersensitivity to any of the study treatments or excipients or to drugs of similar chemical classes.
10. Have a history of any illness, condition, and use of medications that, in the opinion of the Principal Investigator or designee, might confound the results of the study or pose additional risk in administering the investigational agents to the subject or preclude successful completion of the study.
11. Current and/or previous treatment with concomitant medications that are strong or moderate inducers/inhibitors of CYP3A4 (e.g., clarithromycin, ketoconazole, ritonavir, rifampin, etc.) Note: Concomitant medications that are strong or moderate inducers/inhibitors of CYP3A4 should have been stopped at least five (5) half-lives prior to first dosing.
12. Requires treatment with any psychoactive medications, including any anti-seizure medications (with an exception of medications used for short-term treatment of insomnia). Note: SSRI's are allowed if they have adequate stable dose for at least one (1) month prior to dosing.
13. History of malignancy of any organ system (other than localized basal cell carcinoma of the skin or in-situ cervical cancer), treated or untreated, within the past 5 years, regardless of whether there is evidence of local recurrence or metastases.
14. Known history or presence of cardiovascular or cerebrovascular disease such as angina pectoris, myocardial infarction, stroke, transient ischemic attack, peripheral vascular disease.
15. Concomitant use of agents know to prolong the QT interval unless these can be permanently discontinued for the duration of the study.
16. History of porphyria.
17. Women of child-bearing potential, defined as all women physiologically capable of becoming pregnant, unless they are using highly effective methods of contraception during dosing and for 30 days/weeks after stopping of investigational drug. Highly effective contraception methods include:
   Total abstinence from heterosexual intercourse (when this is in line with the preferred and usual lifestyle of the subject). Periodic abstinence (e.g. calendar, ovulation, symptothermal, post-ovulation methods) and withdrawal are not acceptable methods of contraception.
   Female sterilization (have had surgical bilateral oophorectomy with or without hysterectomy), total hysterectomy or tubal ligation at least six weeks before taking investigational drug. In case of oophorectomy alone, only when the reproductive status of the woman has been confirmed by follow up hormone level assessment.
   Male sterilization (at least 6 months prior to screening). For female subjects on the study the vasectomized male partner should be the sole partner for that subject.
   Barrier methods of contraception: condom or occlusive cap (diaphragm or cervical/vault caps) with spermicidal foam/gel/cream/vaginal suppository); placement of an intrauterine device (IUD) or intrauterine system (IUS).
      Note: hormonal contraceptives that are injected or implanted or administered orally or transdermally cannot be considered as effective methods of contraception if taken with study medication.
   Women are considered post-menopausal and not of child bearing potential if they have had 12 months of natural (spontaneous) amenorrhea with an appropriate clinical profile (e.g. age appropriate, history of vasomotor symptoms).
18. Pregnant or nursing (lactating) women, where pregnancy is defined as the state of a female after conception and until the termination of gestation, confirmed by a positive hCG laboratory test.
19. Use of other investigational drugs at the time of enrollment, or within 5 half-lives of enrollment, or within 30 days, whichever is longer; or longer if required by local regulations.

Treatment Arms

Subjects will be assigned to one of the following 2 treatment arms in a ratio of 1:1 at baseline.
Compound (I) arm: up-titration b.i.d. regimen followed by fixed-dose b.i.d. regimen and ending with tapering b.i.d. regimen. See study design section for up-titration and tapering regimen (Tables 1 and 2).
Placebo arm: matching placebo

Efficacy

Clinical efficacy measures (PEG, NRS and TLFB-Opioid) will be collected. In addition, domains suggested within the draft FDA Guideline for opioid reduction will also be assessed by the COWS, OCS, and NOSE, and the DSM-5 defined severity of opioid use disorder.

Analysis of the Primary Endpoint(s)

The purpose of the study is to evaluate the efficacy of Compound (I) in reducing consumption of opioids in chronic low back- and post-surgical pain patients taking between 60 and 100 milligram morphine equivalent dose per day. This will be evaluated by measuring the daily consumption using the TLFB. A subject showing a decrease of the mean weekly consumption between baseline and the last week on treatment of at least 50% will be considered as a responder. PEG assessment at baseline, at week 4, 8, and at end of the study will provide evidence of treatment response over time.

Definition of Primary Endpoint(s)

The primary endpoint of the study is a binary endpoint (responder/non responder). Response is defined as reduction of at least 50% in the mean weekly consumption between baseline (the week preceding and including the baseline visit)) and the last week on-treatment at the maintenance dose (i.e. of 200 mg bid, days 76 to 82) if the subject completes the treatment maintenance period per protocol or any last 7 days on treatment if the subject discontinued treatment before day 82.

Statistical Model, Hypothesis, and Method of Analysis

The number of subjects who are responders in each treatment group is assumed to be a random variable following a binomial distribution Bin(nᵢ, pᵢ) where nᵢ is the number of subjects in treatment group i, and pᵢ is the true underlying proportion of subjects responders in treatment/(i=1 for Compound (I), i=2 for placebo).

Assuming non-informative Beta(1/3, 1/3) priors for the responder rates pᵢ and p₂, the posterior probability of the difference in the rates of responders will be used to calculate Prob (p₁−p₂>0/data) and Prob (p₁−p₂>0.2/data).

Sample Size Calculation

Primary Endpoint(s)

The sample size is assessed based on the chances of “success”, i.e. that the true difference between Compound (I) and placebo responder rates is >0 with least 90% confidence and is >20% with at least 50% confidence at the interim analysis or at final analysis.

The efficacy criteria are formulated in terms of posterior probability statements about the true responder rate difference Δ=p1−p2, where p1 denotes the true responder rate of Compound (I)-treated patients and p2 the true responder rate of placebo-treated patients. The posterior probability distributions for both arms are calculated using a standard binomial probability model with a conjugate noninformative Beta distribution priors Beta(a, a) with a=1/3 for both p1 and p2.

Efficacy Decision Criteria

The decision rule used at final analysis to claim efficacy consists of the following two statements that must be true simultaneously:

1. Posterior probability of Δ>0 is larger than 90%;

2. Posterior probability of Δ>0.20 is larger than 50%.

1.-17. (canceled)

18. A method for treating opioid analgesic tolerance associated with chronic opioid use in chronic pain, in a subject in need thereof, comprising administering to said subject an effective amount of a mGluR5 antagonist.

19. The method according to claim 18, wherein the treatment reverses opioid analgesic tolerance.

20. The method according to claim 18, wherein the mGluR5 antagonist is administered to a patient on an opioid dose of >50 mg oral morphine/day or daily equianalgesic dose of another opioid (i.e. morphine milligram equivalent MME/day), such as a patient on an opioid dose of from 60 to 100 mg oral morphine/day or daily equianalgesic dose of another opioid.

21. The method according to claim 18, wherein chronic pain is associated to an injury (e.g. wound, burn or fracture) or a disease (e.g. diabetes, multiple sclerosis, arthritis, an autoimmune disease or an infection).

22. The method according to claim 18, wherein chronic pain is non-malignant chronic pain.

23. The method according to claim 18, wherein non-malignant chronic pain is chronic back pain, such as chronic low back pain, for example with or without previous spine surgery.

24. The method according to claim 18, wherein the mGluR5 antagonist is administered to an elderly patient.

25. The method according to claim 18, wherein chronic pain is chronic post-surgical neuropathic pain.

26. The method according to claim 18, wherein the mGluR5 antagonist is administered in an immediate-release form or a modified-release form; in particular a modified-release form.

27. The method according to claim 18, wherein the mGluR5 antagonist is administered in the form of a pharmaceutical composition further comprising at least one pharmaceutically acceptable excipient.

28. The method according to claim 18, wherein the mGluR5 antagonist is administered in combination with one or more further pharmaceutical active ingredient.

29. The method according to claim 18, wherein the mGluR5 antagonist is selected from the group consisting of Compound (I), Compound (II), and Compound (III); or in each instance a pharmaceutically acceptable salt thereof.

30. The method according to claim 29, wherein the mGluR5 antagonist is Compound (I) or a pharmaceutically acceptable salt thereof.

31. The method according to claim 30, wherein the mGluR5 antagonist is administered in an amount of from 50 mg/b.i.d to 200 mg/b.i.d, in particular 50 mg/b.i.d., 100 mg/b.i.d or 200 mg/b.i.d., such as 200 mg/b.i.d.

32. The method according to claim 29, wherein the mGluR5 antagonist is Compound (II) or a pharmaceutically acceptable salt thereof.

33. The method according to claim 32, wherein the mGluR5 antagonist is administered in an amount of from 1 mg/day to 100 mg/day.

34. The method according to claim 29, wherein the mGluR5 antagonist is Compound (III) or a pharmaceutically acceptable salt thereof.

35. A combination comprising a mGluR5 antagonist and an opioid.

36. The combination according to claim 35, wherein the opioid is selected from the group consisting of alphamethylfentanyl, alfentanil, buprenorphine, butorphanol, codeine, diacetylmorphine, dihydrocodeine, dihydroetorphine, dihydromorphine, ethylmorphine, etorphine, fentanyl, hydrocodone, hydromorphone, L-acetylmethadol, levorphanol, methadone, meperidine, morphine, nicomorphine, normethadone, noroxycodone, normorphine, norlevorphanol, oxycodone, oxymorphone, phenazocine, propoxyphene, remifentanil, tramadol, thebaine, tapentadol, levorphanol, sufentanil, pentazocine, carfentanyl, ohmfentanyl, nocaine, ketobemidone, allylprodine, prodine, dextropropoxyphene, dextromoramide, benzitramide, piritramide, dipipanone, loperamide, diphenoxylate, nalbuphine, levomethorpha, dezocine, lefetamine and tildine; in particular, the opioid is selected from the group consisting of hydrocodone and oxycodone.

37. The combination according to claim 35, wherein the mGluR5 antagonist is selected from the group consisting of Compound (I), Compound (II), and Compound (III); or in each instance a pharmaceutically acceptable salt thereof.

38. A method for treating opioid analgesic tolerance associated with chronic opioid use in chronic pain, the treatment reversing opioid analgesic tolerance, in a subject in need thereof, comprising administering to said subject a pharmaceutical composition comprising an effective amount of a mGluR5 antagonist and at least one pharmaceutically acceptable excipient.

39. A method for treating opioid analgesic tolerance associated with chronic opioid use in chronic pain, in a subject in need thereof, comprising administering to said subject a pharmaceutical combination comprising an effective amount of a mGluR5 antagonist and at least one further pharmaceutical active ingredient.
